(19) United States
(12) Patent Application Publication
(10) Pub. No.: US 2017/0340254 A1
(43) Pub. Date: Nov. 30, 2017

(54) Real-time blood detection system

(71) Applicants: Alice McKinstry Davis, Palm Bay, FL (US); Dennis Willard Davis, Palm Bay, FL (US); Russell Denning Davis, Miami, FL (US)

(72) Inventors: Alice McKinstry Davis, Palm Bay, FL (US); Dennis Willard Davis, Palm Bay, FL (US); Russell Denning Davis, Miami, FL (US)

(21) Appl. No.: 15/731,343

(22) Filed: May 31, 2017

Related U.S. Application Data
(63) Continuation-in-part of application No. 14/121,598, filed on Sep. 22, 2014, now abandoned.
(60) Provisional application No. 61/960,643, filed on Sep. 23, 2013.
(60) Provisional application No. 61/997,886, filed on Jun. 12, 2014.

(51) Int. Cl. A61B 5/15; A61L 15/16; A61B 5/00

(52) U.S. Cl. CPC A61B 5/15087; A61B 5/150045; A61B 5/6802; A61L 15/16; A61B 5/0022; A61B 5/4337

(57) ABSTRACT

Disclosed is a system for real-time detection and annunciation of blood associated with menstruation and surgical wounds. The system comprises a real-time, wide area blood detector, communication means for relay of blood detection information, and annunciation means to inform the user of the emanation of blood. Various system embodiments include local and remote as well as covert and non-covert annunciation to users or medical personnel, various forms of real-time blood detection sensors, blood analysis capability, and smart bandage telemetry.

### CROSS-REFERENCE TO RELATED APPLICATIONS

This application is a continuation-in-part of U.S. Nonprovisional application Ser. No. 14/121,598, filed Sep. 22, 2014, now abandoned and claims the benefit of U.S. Provisional Application No. 61/960,643, filed Sep. 23, 2013 and U.S. Provisional Application No. 61/997,886, filed Jun. 12, 2014.

### FIELD OF DISCLOSURE

This disclosure broadly relates to a system for the real-time detection of blood. More specifically, the invention relates to such a system for the discreet annunciation to the user of the presence of blood associated with menstruation and analysis of such blood or clinical annunciation of blood emanating from a wound.

### BACKGROUND

Women continue to suffer from the uncertainty concerning menstrual flow at the onset of a menstrual period. Specifically, the prediction of menstrual flow is imprecise based on calendar considerations of the timing of the last period. This becomes exacerbated when women enter peri-menopause with wide ranging fluctuation in the intervals and durations of menstruation. Often, women are faced with having to wear feminine hygiene products well in advance of onset of flow or suffer the consequences of not having done so, such as blood staining of undergarments, embarrassing blood spotting of outerwear, and staining of bed clothing and bed covers. Current options for women comprise the use of tampons, menstrual pads, or pantyliners (or panty shield). Tampons are less used today given issues surrounding infection and toxic shock syndrome. Various menstrual pad geometries seek to mitigate the problem of blood leakage while minimizing discomfort associated with a bulky pad. These approaches include use of thickened absorbing material around the pad perimeter, a padded circumference around the core of the pad, improved fluid absorbing materials, etc. However, none of these innovations can be considered full proof means of preventing blood leakage.

Because use of menstrual pads can be uncomfortable, lead to chafing, can promote yeast infections, and are an expense, it is desirable to avoid their use when it is not necessary. Hence, there is motivation to wear menstrual pads for the least possible amount of time by using thin pantyliners in advance of the need for menstrual pads. When to transition from pantyliner to menstrual pad is the critical issue and is a source of significant anxiety for many women. Visual inspection of the pantyliner often is too late to prevent staining of clothing. Therefore, a need exists for a non-visual means to annunciate to the user of a pantyliner the first presence of blood.

Also, there exists a need for a real time indication that a menstrual pad is saturated so that the same issues associated with spotting can be avoided. Menstrual bleeding can commence during sleep, so means to wake the user and inform them of this event would be useful.

Conventional methods of addressing the blood sensing problem include various ways to infer the presence of blood by sensing moisture or various blood-borne analytes. The false alarm rate associated with these measures can be dramatically reduced by increasing the specificity of the indicator. Annunciation means using sensors confined to the hygiene pad, have included thermochemical and visual; it would be useful to improve the reliability and convenience of annunciation by other methods that exploit skin contact. Radiofrequency means of alerting the user have made use of direct electrical connection between sensors and communication devices. Improved convenience would obtain through avoidance of such connections.

Additionally, it would be advantageous to perform micro diagnostics on menstrual blood, for general health reasons, since it is available monthly as a natural effluent of the body. Sometimes during a period, women experience odor emanating from a pad even before it is in a saturated state. In some cases, they may not detect the odor when others nearby might. Hence, having a sensor to detect this odor, or the bacterial growth causing it, would be advantageous in alerting a menstrual pad user to replace the pad.

Finally, this same type of sensing and portable analysis would be beneficial in assessing the status of skin and general health through measurement of perspiration and health markers contained in perspiration, type and quantity of surface bacteria, and other measures of skin health.

### Hygiene Articles

Examples of hygiene garments and feminine hygiene articles that include sensors of various kinds and even RF communication means are provided below:

U.S. Pat. No. 5,728,125 to Salinas, discloses a menstrual detector comprising a sanitary napkin of reduced dimensions, formed by a thin layer of absorbent material, and within the interior of the absorbent material a small cavity containing a temperature-sensitive reactive chemical product that can respond by turning cold when it comes into contact with and dissolves in a menstrual flow.

U.S. Pat. No. 6,348,640 to Navot, et al. discloses an electrically conductive wetness sensor as part of a tampon that enables transmission from a radio frequency identification transmitter.

U.S. Pat. No. 6,570,053 to Roe, et al. discloses a disposable wearable article that incorporates a sensor to detect any of a number of physiological inputs that correlate with an impending elimination of bodily waste from the wearer.

U.S. Pat. No. 6,713,660 to Roe, et al. discloses a disposable, wearable article that incorporates a sensor that detects a target biological analyte in bodily waste or on the wearer's skin.

U.S. Pat. No. 6,921,647 to Kritzman, et al. discloses a secretion-monitoring article for identifying a secreted biological fluid having a body with an absorbent material and least one pH determining member and a reagent associated with the absorbent material. The goal is detection of infectious pathogens.

U.S. Pat. No. 7,176,344 to Gustafson, et al. discloses a disposable sensor absorbent structure for detecting wetness comprising at least one absorbent layer and at least one sensing device comprising a magnetoelastic film.

U.S. Pat. No. 8,248,249 to Clement, et al. discloses an RFID (radiofrequency identification) tag and a system and method involving a plurality of RFID tags. Each RFID tag is attached to an object on which the presence of a predefined fluid is monitored. In a first state, (absence of the monitored fluid), the tag acts as a passive RFID tag, and the information it holds can be read with a proximity reader. In a second state, whenever the monitored fluid appears on the tagged object, a fluid activated battery generates the electrical power which is used to power the RFID tag.

U.S. Pat. No. 8,492,609 to Ecker, et al. discloses a feminine hygiene article with improved visual indication of the core absorbing area of the article. Side leakage, i.e. the leakage of previously absorbed liquid through the side edges of the core of the articles, is a common problem in the field of feminine hygiene articles. The invention seeks to provide the visual identity of the core absorbing area so that easy determination can be made as to when the maximum capacity of the absorbent core of the article has been approached.

### Blood Sensors

Sensors for the real-time detection of blood are limited in number. One example is provided in U.S. Pat. No. 8,048,045 to Engvall which discloses a photonic means of detecting blood flow or leakage from a wound or IV by attenuation of fiber conducted light when the fiber contacts blood. Most sensors dealing with blood, address detection of certain constituents of blood rather than the presence or absence of blood itself. The present disclosure addresses this under developed technology area. In addition, “lab-on-a-chip” (LOC) type blood sensors used to characterize health state through measurement of various blood chemistries are included in an embodiment of the disclosure for a smart sanitary pad or smart bandage with telemetry.

### Communications Technology

Low power radio frequency communication technologies are particularly relevant to the system concept disclosed herein. These include methods for use in the FCC-designated unlicensed ISM bands as well as low power infrared communication techniques. The technologies associated with small RF transmitters, oscillators, receivers, and transceivers are relevant to this disclosure. In this application, on-body sensing will occur, consequently there can be large radio frequency propagation losses associated with communicating with a body-mounted RF device. Apart from using large transmit powers and high receiver sensitivity implementations, various methods can be used to achieve high processing gain to overcome such losses; these include spectrum spreading methods such as direct sequence, frequency hopping, and ultra wideband approaches. Also, antenna technologies that can provide gain in small antenna volumes, such as retro-directive arrays, and antennas that exhibit high radiation efficiency in small volumes, such as chip antennas with associated ground planes of limited size, are pertinent. Antenna diversity and pattern diversity can be accomplished by switching connections among different antennas to improve link margin.

Various protocols and IEEE standards are relevant to this disclosure including but not limited to Wireless Body Area Networks (802.15.6), Wireless LAN (802.11a/b/g/n), Bluetooth and Bluetooth Low Energy (802.15.1), Zigbee (802.15.4), WiFi Direct uses of 802.11, and Medical Body Area Networks (802.15.4j). Technologies pertinent to this disclosure include those for remote keyless entry (RKE), short range devices (SRDs), and RFID. Emerging standards for the 60 GHz ISM band will be relevant, as well.

### Bluetooth Transceivers

Bluetooth technology transceivers and protocols, especially the more recent protocol updates including Bluetooth Low Energy (LE), provide particularly attractive communication functionality for the presently-disclosed system. Sufficient communication range is provided at low power consumption levels necessary for portable and even disposable devices. Currently, there is a proliferation of Bluetooth-enabled personal appliances that exploit either transceiver or beacon modes of operation. WiFi Direct is an emerging point-to-point protocol exploiting the IEEE 802.11 standard and may provide significant competition for Bluetooth in the future.

### Radio Frequency Identification (RFID)

Technology related to RFID and medical monitoring transducer links is relevant to the present disclosure. In recent years, there has been an explosion of RFID development activity. Noteworthy are the patents to Gentag which disclose RFID technology for telemetry of medical parameters from the surface of a person to a nearby receiver. The close proximity (up to 4 inches of transponder-receiver separation) implementations use near field communication (NFC) at 13.56 MHz.

The body-worn glucose monitoring and insulin dispensing system, the Medtronic MiniMed Paradigm® REAL-Time System is another example of body-borne sensing combined with communication technology. The system comprises a glucose sensor (having a sensing needle implanted through the skin) and an associated transmitter that relays data to a small body-worn insulin pump. The system operation is in the 915 MHz band.

### RFID Tags

As suggested above, RFID technology is rather mature, with internationally-ratified standards that govern the use of such technology. There are three categories of RFID Tags, active, passive, and semi-passive, each of which is relevant to the present disclosure.

### Passive RFID Tags

Among types of passive transponders are RFID tag technologies. The most relevant to the present disclosure are those that exhibit efficient radio frequency returns. Commercially-available passive and semi-passive tags modulate electromagnetic energy backscattered to the interrogator transceiver. Hence they provide an economical approach to sensor communication. High processing gain implementations include surface acoustic wave (SAW) filter correlators for UWB signals such as chirp waveforms. Various passive tags have been implemented with sensors to provide remote sensing capability in the commercial setting.

### Conclusion

No prior art has provided a real time, low false alarm rate indication of the onset of bleeding using a simple thin disposable fabric-based sensor. Nor has there been provided a real time indication to a feminine hygiene pad wearer of a blood saturated state of the pad so that said pad can be replaced. Also, there does not exist a feminine hygiene product with ability to sense and alert the user to bacterially-caused odor. Further, the inventors are not aware of an inexpensive, disposable real-time indicator system for wound blood leakage detection.

The system of the present invention comprises a very low cost disposable communication and sensor combination that can be used with conventional communication devices or low cost reusable electronics that alert the user of the incidence and status of blood flow by either covert or non-covert annunciation.

### SUMMARY OF THE DISCLOSURE

The present disclosure concerns methods and a system for the detection and annunciation of the emanation of blood flow from the body whether from a wound, surgical incision, or menstruation using real-time blood detection sensors. The system for blood detection comprises a) a real-time blood detection sensor; b) first radio frequency communication means electronically connected to the real-time blood detection sensor; c) second radio frequency communication means; and d) annunciation means, the blood detection sensor providing a signal to the first radio frequency communication means upon detection of blood, the first radio frequency communication means communicating with the second radio frequency communication means so as to convey blood detection information, and the annunciator annunciating blood detection upon receipt of signal from the second radio frequency communication means.

The system concept comprises a few main components with alternative system architectures depending on the specific implementation. In a first configuration, notification of bleeding is achieved by a blood detection communication from a first RF communicator attached to a blood sensor to a second RF communicator attached to an annunciator. Alternatively, the second RF communicator and annunciator functions can be separated to support a more covert mode of operation. In this case, the standalone second RF communicator would communicate with the first RF communicator and, upon blood detection, would communicate with a small wrist-worn or body-worn receiver connected to a vibratory annunciator transducer or visual display.

A few options exist for RF communicator components of the system. In one option, the first RF communicator attached to the blood sensor can be a transceiver and the second RF communicator also can be a transceiver. The second transceiver communicates with the first transceiver periodically and upon sensor detection of blood, causes an annunciator to issue an audible annunciation, such as a ring tone. The second transceiver and annunciator can be carried off-body in a purse or pocket.

In another option, the first RF communicator attached to the blood sensor can be a transponder and the second RF communicator can be an interrogator. The interrogator is a small battery-powered transceiver that queries the transponder periodically and causes an annunciator to issue an audible annunciation, such as a ring tone, upon sensor detection of blood. This interrogator and annunciator can be carried off-body in a purse or pocket. In yet another option, the first RF communicator is a beacon that transmits a signal indicative of blood detection to the second RF communicator in the form of a beacon receiver.

Alternative configurations discussed in the Detailed Description vary the on-body and off-body locations of various system components, including covert or non-covert annunciation, processing, and data relay means. A number of variations of implementation are disclosed including different types of blood sensing mechanisms, transceiver means that are either half duplex or full duplex, transponder means comprising active, passive, and semi-passive RF-based sensor transponders, as well as the use of beacons.

In a first embodiment of the present disclosure addressing menstruation, the worry associated with not wearing hygiene products in advance of actual menstruation is mitigated. This is achieved by sensing of the slightest release of blood and surreptitiously annunciating this detection to the woman so that appropriate action can be taken.

The first implementation of the embodiment addressing the problems associated with menstrual sensing comprises an inexpensive disposable sensor and a disposable first RF communicator in concert with a separate reusable second RF communicator and associated annunciator. The sensor is installed easily in an undergarment, is small and unobtrusive, provides no sensation concerning its presence, and is inexpensive. A simple, thin, small blood detection sensor is provided that can be placed in the crotch of undergarments in the form of a thin fabric insert or pantyliner to detect the first onset (including only spotting of blood) of a monthly menstrual flow in an area of several square inches proximate to the source of blood flow. Upon notification of blood presence to the wearer, this sensor (pantyliner) can be disposed of and replaced with a feminine hygiene pad.

In a second embodiment of the disclosure, saturation of a hygiene pad is sensed and annunciated prior to blood broaching the boundaries of the pad, permitting the wearer of the pad to attend to the situation before undesirable leakage occurs. In this embodiment, the sensor is manufactured into the hygiene pad. The pad is manufactured to contain the same kind of disposable blood sensor, but in this case, the sensing is not across a wide area, but limited to the peripheral locations on the pad in order to detect a blood-saturated state of the pad prior to blood leakage from the pad. The associated, disposable RF communicator electronics may be likewise part of the pad.

The annunciation for both the aforementioned embodiments can be of sufficient intensity to wake users from sleep to attend to the need to replace pantyliner or menstrual pad.

In a third embodiment of the disclosure, a pantyliner contains a blood detection sensor, disposable power source, such as a paper battery, disposable interface electronics, and disposable miniature vibrating transducer for annunciating menstrual blood detection.

In a fourth embodiment of the disclosure, a pantyliner contains chemicals that create a temperature change when exposed to blood (after the fashion of U.S. Pat. No. 5,728,125) and a temperature sensor with radiofrequency communications means. The temperature change alert is communicated to an annunciator to inform the user of the onset of menstruation. Also, it would be possible to include two temperature sensors, one to perform a reference measurement and the second to actually sense temperature change due to the aforementioned chemical reaction.

In a fifth embodiment of the disclosure, the pad is equipped with a disposable micro diagnostics capability in order to perform selected blood tests which are relayed to a receiver by disposable RF communicator electronics. This embodiment invokes LOC technology for analysis of blood constituents.

It is important to insure that sutured wounds and surgical incisions do not bleed or hemorrhage. In a sixth embodiment, the basic components of this same system can be used in the detection of blood from wounds. The system can telemeter the instance of deleterious bleeding of wounds to health care staff. A thin, cloth-based, wide-area sensor is either applied to the wound under the wound dressing or is made part of the wound dressing. A distribution of sensing areas can be used to quantify the extent of bleeding. Detection of blood associated with the wound is relayed for alarm annunciation or telemetry to a central area such as a nurse's station. The distribution of sensing areas can be annotated on an indicator or annunciator with specific areas of bleeding detection indicated accordingly; this could provide a bleed map to nurses' stations.

A final, seventh embodiment comprises a “smart bandage” with telemetry capability. Sensors incorporated in the bandage can detect various pathogens, when they transition from commensal to infectious states, status of healing wound tissue, analysis of perspiration markers, etc. This information can be logged in local memory or transmitted to a receiver remote to the bandage, whether on the body or in an off-body location.

### Definitions

Real-time, relatively thin, wide area blood detection sensor—concerns a sensor that can detect the presence of small amounts of blood within the timeframe of seconds, anywhere over a contiguous area of several square inches in proximity to the area of blood emanation (the groin, in the case of menstrual blood) and can provide a change to a sensible physical parameter that can be transduced into an electrical signal. Hence various blood components and combinations of such components can be the basis for sensing blood presence. These may include, but are not limited to albumin, hemoglobin, immunoglobulins, globulins, heme, ferritin, iron, transferrin, glucose, A1C, fibrinogen, cholesterol, cortisol, and hormones. Chemiresistor sensors are leading candidates for the blood detection sensor, but other devices can be used that alter a sensible electrical parameter such as impedance or voltage. This would include impedimetric (ex ChemFET), amperometric, potentiometric, or even optically-based sensors using micro LEDs and detectors. Combinations of simpler sensors such as moisture with viscosity sensors can be used to create a blood sensor that discriminates against non-blood liquids. The sensing mechanism within the wide area sensor must exhibit the ability to detect a single droplet of blood deposited anywhere within an area of several square inches. This sensor is thin enough to be made part of a pantyliner insert, and hence should be not much thicker than a few sheets of thin fabric. The objective is to have a pantyliner insert comprising such a blood detector, along with transceiver electronics, disposable battery, and antenna that is not much thicker than a conventional pantyliner (such as a Kotex Barely There Panty Liner) which may be 1 to 2 millimeters thick. Hence the definition of “relatively thin” is a thickness in the range of a few or more millimeters. To achieve this thickness goal, thin paper or fabric substrates can be used to support ink-jet printed or bonded blood sensors and electronics. Alternatively, to reduce thickness, some components of the presently disclosed system can be made separate from the pantyliner, such as the antenna.

Annunciator—relates to the means for annunciation of blood presence to the user of the disclosed system. The annunciator may comprise an audible signal (such as a smart phone ring tone) generator or a tactile, body-worn vibration based transducer. The audible annunciator typically would be co-located with the a transceiver (or interrogator) that is remote to the user's body whereas the tactile annunciator would be placed in contact with the user's body and would generate an annunciation when prompted by either a transceiver, an interrogator, or an annunciation receiver. Additionally, the annunciator may be contained in a device located remote to the body, such as a smart phone. To alert users during sleep, the annunciator output can be made sufficiently loud or provocative to wake the user. In an embodiment including an annunciation receiver, the annunciator also may take a visual form such as an LCD or LED display.

Data relay means—refers to the means by which blood detection information is provided by the interrogator to a destination beyond the interrogator. For wound bleed detection, not only the bleed event but information about location and extent of bleeding may be relayed. Destinations for such information include data terminals, data logging means, and nurses' station displays. Data relay can be achieved by wired, or wireless RF or infrared links.

Annunciator receiver—refers to receiver means that receives a blood detection signal from the interrogator and activates a body-worn annunciator, typically a vibration transducer, but may include visual annunciation such as an LCD or LED display.

Processor—refers to the means for converting sensed blood information (blood detection or blood marker analysis data) or skin-related information (infection information, chemical markers, etc.) in the case of smart bandages, into electronic signals or into annunciation events. In the case of wound monitoring, the processor can convert the blood information into data for transmission, recording, or annunciation. The processor is connected to the blood sensor and transduces the signal from the blood sensor into digital data and into an alert signal that may be digital or analog in nature. The processor may be a standalone article of medical equipment or it may provide the detected bleed information to another destination, such as a nurses' station display, remote devices linked by Internet, or even cell phone infrastructure, through data relay means.

Mircrocontroller unit (MCU)—refers to a system on a chip with one or more processor cores with memory and programmable input/output peripherals. An MCU is often part of the interface between various sensors and Bluetooth transceivers or Bluetooth sensor beacons and is often contained in Bluetooth IC chips, such as sensor chips, exhibiting highly integrated functionality in addition to communication.

Bluetooth Microcontroller—refers to a system on a chip which includes processors as well as Bluetooth transceiver functionality.

RF communicator—refers to various RF communication devices that can be part of the system configuration. This includes a a) transceiver, b) transponder, c) beacon, d) interrogator, which communicates with a transponder, and e) beacon receiver which communicates with a beacon:
Transceiver—relates to radio frequency means of communication in either half duplex or full duplex mode. Transceivers comprise both receiver and transmitter functions. Two such transceivers are necessary to compose a communications link. A prime example of a transceiver to be used in the presently disclosed system is a Bluetooth LE device that is connected to a blood detection sensor. This transceiver would communicate the detection of blood to another transceiver in proximity such as a Bluetooth-enabled smart phone that would annunciate the blood detection to the user. The Bluetooth protocol permits ease of device (transceiver) pairing and bonding. Alternatively, other transceiver devices could be used.
Transponder—relates to radio frequency means of responding to an interrogation from the interrogation transceiver (interrogator). The transponder may be active, passive, or semi-passive and hence, may operate without a battery, may be battery-powered or battery-assisted, and may harvest ambient electromagnetic energy or interrogator transmission energy. In some instantiations, the blood detection sensor and transponder are intimately coupled such as when the sensor alters the response of a passive transponder. Also, the transponder may include logic and processing functions as, for example, in the EM4325 RFID chip, which includes temperature sensor, RF interface, and I/O control along with memory management. The transponder can be based on custom designs or exploit commercially available RFID tag or sensor tag technology.
Beacon—refers to a transmitter connected to a blood detection sensor. At a minimum, it transmits a signal to a beacon receiver when blood is detected by the blood detection sensor. It may also periodically transmit a signal indicative of proper working condition of the beacon.
Interrogator (interrogation transceiver)—relates to radio frequency means of interrogating or querying a transponder. The primary information obtained by query is the presence or absence of blood indicated by the blood sensor. Additional information obtained by the interrogator may include transponder security information such as transponder identification, self-test, or other information. It may have processing and memory functionality as well to record such information as menstrual intensity, period intervals, and durations. In the system embodiment that includes LOC type sensing for menstrual blood analysis, additional processing may be performed for this analysis purpose.
Beacon receiver—refers to a receiver that receives a signal indicative of blood detection from a beacon. The beacon receiver may include processing and memory functionality for the same purposes as an interrogator.
Beacon transceiver—refers to the combination of a receiver that receives a signal indicative of blood detection from a beacon and a transmitter that relays this information to a an annunciation receiver that may or may not be body-worn.

Pantyliner insert—refers to the present blood detection system that is either manufactured into a pantyliner or is installable in a commercial off-the-shelf pantyliner.

### BRIEF DESCRIPTION OF DRAWINGS

FIG. 1A is a functional block diagram of a blood detection system comprising a body-borne sensor and transceiver.

FIG. 1B is a functional block diagram of a blood detection system comprising a body-borne sensor and transceiver and a remote transceiver and annunciator.

FIG. 1C is a functional block diagram of a body-borne blood detection system comprising a sensor, a first transceiver, a second transceiver, and an annunciator.

FIG. 2A is a functional block diagram of a blood detection system comprising a body-borne sensor and transponder and a remote interrogator and annunciator.

FIG. 2B is a functional block diagram of a blood detection system comprising a body-borne sensor, transponder, annunciation receiver, and annunciator, and a remote interrogator.

FIG. 2C is a functional block diagram of a body-borne blood detection system comprising a sensor, transponder, interrogator, and annunciator.

FIG. 2D is a functional block diagram of a body-borne blood detection system comprising a sensor and annunciator.

FIG. 3A is a functional block diagram of a blood detection system comprising a body-borne sensor and beacon, a remote beacon receiver and annunciator.

FIG. 3B is a functional block diagram of a blood detection system comprising a body-borne sensor, beacon, annunciation receiver and annunciator, and a remote beacon transceiver.

FIG. 3C is a functional block diagram of a body-borne blood detection system comprising a sensor, beacon, beacon receiver and annunciator.

FIG. 4A is a functional block diagram of a blood detection and analysis system comprising a body-borne sensor and transponder, and a remote interrogator with data relay means to various destinations.

FIG. 4B is a functional block diagram of a blood detection and analysis system comprising a body-borne sensor and processor, and a remote data relay means to various destinations.

FIG. 4C is a functional block diagram of a blood detection and analysis system comprising a body-borne sensor, and a remote processor interrogator with data relay means to various destinations.

FIG. 5 is a functional block diagram of a blood detection and analysis system comprising a body-borne sensor and beacon, and a remote beacon receiver with data relay means to various destinations.

FIG. 6A is a pictorial diagram of the real-time menstrual blood sensor used in concert with a body-borne transceiver to communicate with a remote transceiver.

FIG. 6B is a plan view diagram of a pantyliner insert containing components of FIG. 6A.

FIG. 6C is an exploded diagram of the pantyliner insert of FIG. 6B.

FIG. 6D is a functional block diagram showing the electrical connectivity of the components made part of the pantyliner insert of FIG. 6B.

FIG. 7A is a pictorial diagram of the real-time menstrual blood sensor used in concert with a remote interrogator or receiver.

FIG. 7B is a plan diagram of a pantyliner insert with menstrual blood sensor and RF communication means.

FIG. 7C is a plan diagram of a pantyliner insert with menstrual blood sensor and vibrating annunciator.

FIG. 7D is a plan diagram of a pantyliner insert with menstrual blood sensor, lab-on-a-chip blood analysis sensor, and RF communication means.

FIG. 8 is a pictorial diagram of the real-time menstrual blood sensor used in concert with a remote interrogator or receiver that relays alert information to a body-worn annunciation device.

FIG. 9 is a plan diagram of a menstrual pad with edge sensors to alert the user to the saturated state of the pad.

FIG. 10A is a pictorial diagram of the real-time menstrual blood sensor that communicates by RF means with a remote body-worn annunciator.

FIG. 10B is a pictorial diagram of battery insertion in the interrogator-annunciator module used in the system of FIG. 8a.

FIG. 11A is a pictorial diagram of the real-time menstrual blood sensor that communicates by electrical connection means with a remote body-worn annunciator.

FIG. 11B is a plan diagram of the menstrual blood sensor in a pantyliner with electrical connection to an annunciator.

FIG. 12 is a pictorial diagram of the real-time blood sensor for wound monitoring that communicates by electrical connection means with a remote annunciator.

FIG. 13 is a pictorial diagram of the real-time blood sensing system for wound monitoring that telemeters data to remote devices.

FIG. 14 is a pictorial diagram of a first implementation of a chemiresistor blood sensor.

FIG. 15 is a pictorial diagram of a second implementation of a chemiresistor blood sensor.

FIG. 16A is a notional geometry of an array of chemiresistors spanning the blood deposition area of a pantyliner insert.

FIG. 16B is the electrical topology of a row of the chemiresistors of FIG. 16A.

FIG. 16C is a diagram of a circuit for injection of a precision current into the chemiresistors of

FIG. 16A to product a blood detection voltage.

FIG. 17 is a pictorial diagram of an optically-based blood sensor.

FIG. 18 is a functional block diagram of a Bluetooth Low Energy microcontroller chip exhibiting an analog sensor interface.

FIG. 19 is a schematic diagram of a harmonic-generating transponder circuit.

FIG. 20A is a schematic diagram of an RFID sensor tag (EM4325 part) used with an impedimetric sensor.

FIG. 20B is a schematic diagram of an RFID sensor tag (EM4325 part) used with direct connection to an amperometric sensor.

FIG. 20C is a schematic diagram of an RFID sensor tag (EM4325 part) used with a transimpedance amplifier interface to an amperometric sensor.

FIG. 21A is a plan and pictorial diagram of a compact menstrual blood sensing pantyliner using a loop antenna.

FIG. 21B is a plan diagram of a compact menstrual blood sensing pantyliner using an antenna design specific to body mounting applications.

FIG. 22A is a plan diagram of an extended menstrual blood sensing pantyliner using a loop antenna.

FIG. 22B is a plan diagram of an extended menstrual blood sensing pantyliner using an antenna design specific to body mounting applications.

FIG. 22C is a pictorial diagram of garment positioning of an extended menstrual sensing pantyliner.

FIG. 23A is a plan diagram of an antenna patch applique.

FIG. 23B is a pictorial diagram of a first placement of an antenna patch applique within an undergarment.

FIG. 23C is a pictorial diagram of a first placement of an antenna patch applique within an undergarment.

FIG. 24 is a pictorial diagram of a smart bandage with the capability of communicating with various receivers.

### DETAILED DESCRIPTION

A system level description of the invention is followed by detailed subsystem and component descriptions.

### System Configuration

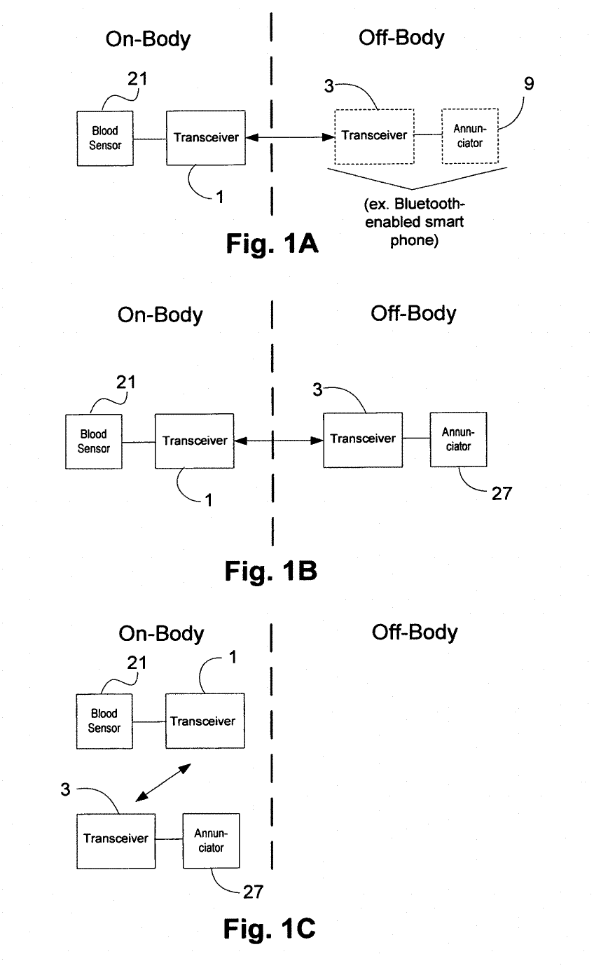

Presently disclosed is a system for detection and annunciation of the presence of blood from menstrual flow or a wound. Reference is made to the functional block diagrams of FIG. 1 depicting a first set of alternative system architectures which may be applicable to both menstrual and wound sensing. In these system architectures, a first RF communicator which is attached to the blood sensor is a transceiver and the second RF communicator which is attached to the annunciator also is a transceiver. The dotted separation line establishes which components are body-mounted and which are not. In the first configuration of FIG. 1A is depicted the use of a blood sensor 21 electrically-connected to transceiver 1; both are placed on the user's body. The transceiver 1 is in RF communication with a remote transceiver 3 and communicates an indication of bleeding provided by blood sensor 21. The transceiver 3 is in communication with annunciator 27 that alerts the user of bleeding. In application to menstrual sensing, this configuration is representative of the use of a pantyliner-type insert that includes a disposable blood sensor 21 and transceiver 1. In a preferred implementation, the transceiver 3 could be a Bluetooth-enabled device such a smart phone or tablet computer and the annunciator 27 could be the mechanism for providing a visual or audible alert from this device. Alternatively, as depicted in FIG. 1B, the transceiver 3 can be a standalone dedicated transceiver carried in the purse or pocket with the annunciator 27 being a ring tone or other audible generator.

Corresponding to FIG. 1C is the combination of blood sensor 21 and transceiver 1 as in the configurations of FIGS. 1A, and B, but in which the transceiver 3 and annunciator 27 are worn on the user's body, most likely in the form of a skin patch with the annunciator 27 comprising a vibrational transducer.

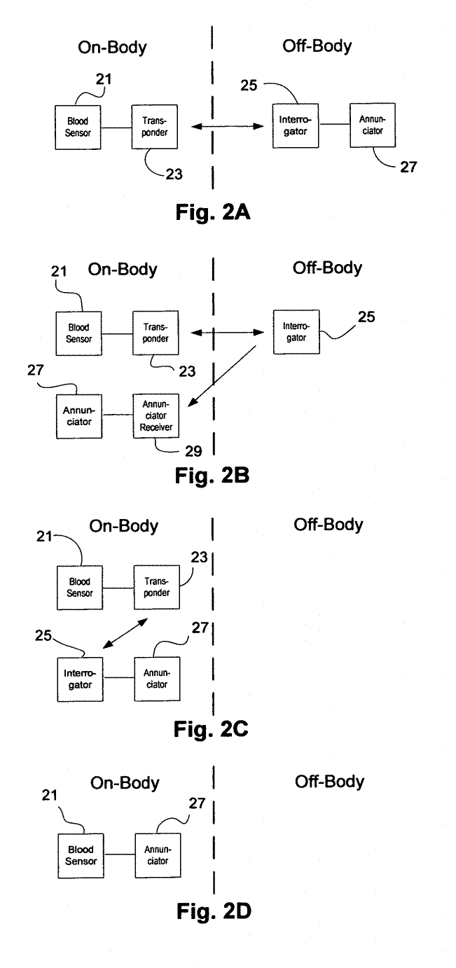

Reference is made to the functional block diagrams of FIG. 2 depicting a first set of alternative system architectures which may be applicable to both menstrual and wound sensing. In these system architectures, a first RF communicator which is attached to the blood sensor is a transponder and the second RF communicator which is attached to the annunciator is an interrogator. The dotted separation line establishes which components are body-mounted and which are not. In the first configuration of FIG. 2A is depicted the use of a blood sensor 21 electrically-connected to transponder 23; both are placed on the user's body. The transponder 23 is in RF communication with a remote interrogator 25 and responds to queries from the interrogator 25 with an indication of bleeding provided by blood sensor 21. The interrogator 25 is in communication with annunciator 27 that alerts the user of bleeding. In application to menstrual sensing, this configuration is representative of the use of a pantyliner type insert that includes a disposable blood sensor 21 and transponder 23. The interrogator 25 can be a standalone transceiver carried in the purse or pocket with the annunciator 27 being a ring tone or other audible generator. Alternatively, the interrogator 25 could be a Bluetooth enabled device such a smart phone or tablet computer and the annunciator 27 could be the mechanism for providing a visual or audible alert from this device. FIG. 2B represents a configuration in which the blood sensor 21, transponder 23, and interrogator 25 interoperate as in FIG. 2A, but in which the annunciator 27 is worn on the user's body in the form of a vibrating transducer. Upon detection of blood, the interrogator 25 sends a signal to body-worn receiver 29 and a vibrational alert is generated. The annunciator transducer and associated receiver can be in a wristband or body patch, not unlike a nicotine patch. Corresponding to FIG. 2C is the combination of blood sensor 21 and transponder 23 as in the configurations of FIGS. 1a, and b, but in which the interrogator 25 and annunciator 27 are worn on the user's body, most likely in the form of a skin patch with the annunciator 27 comprising a vibrational transducer. A minimal configuration is depicted in FIG. 2D in which the blood sensor 21 is connected directly to the body-worn annunciator 27. The issues associated with this configuration have to do with the type of battery power necessary and transducer placement for use in menstrual sensing. Also, the architecture of FIG. 2D is useful for addressing the needs of wound monitoring or hemodialysis access sites in which case blood sensor 21 can be made part of a surgical dressing or other wound applique and a power supply may be included to energize the annunciator and associated electronics.

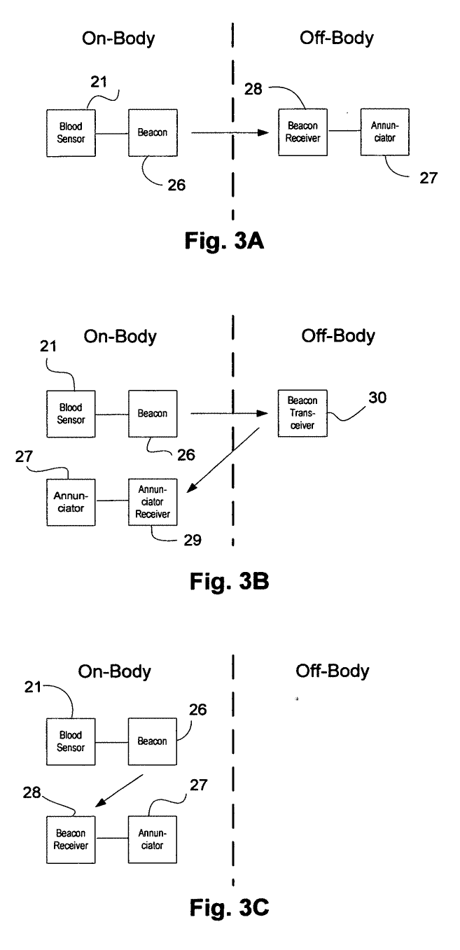

The functional block diagrams of FIG. 3 depict another set of alternative system architectures which may be applicable to both menstrual and wound sensing. In these system architectures, a first RF communicator which is attached to the blood sensor is a beacon 26 and the second RF communicator which is attached to the annunciator is a beacon receiver 28. Hence, FIGS. 3A through 3C correspond to FIGS. 2A through 2C with this replacement of RF communicator components.

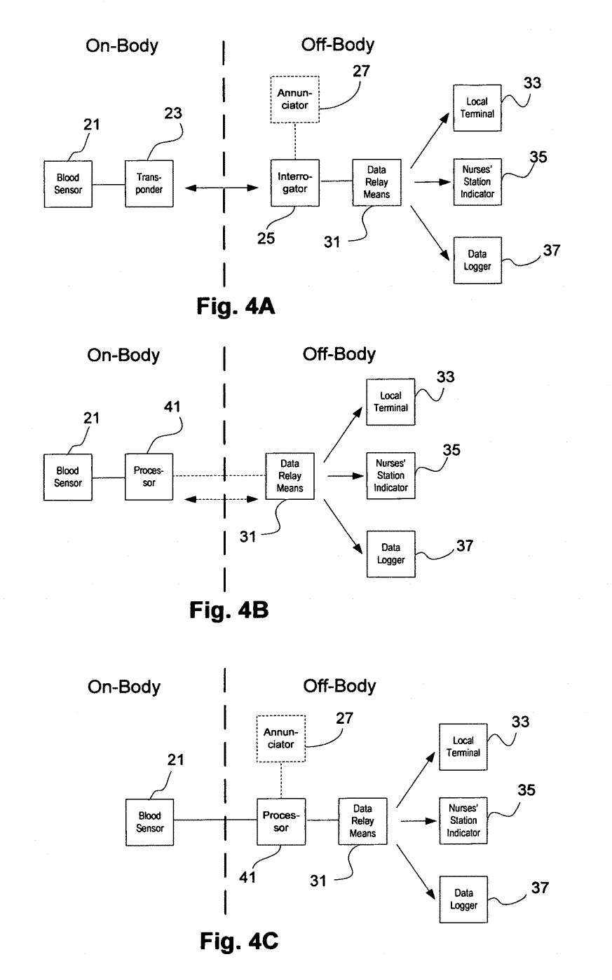

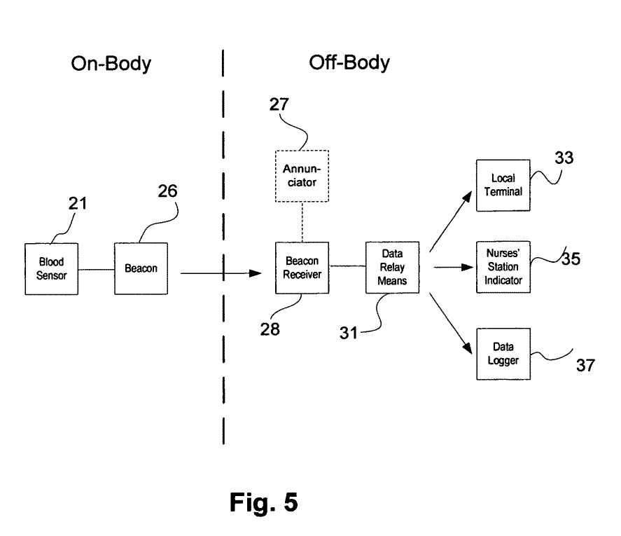

In FIG. 4 are depicted system architectures that are more applicable to wound sensing than menstrual sensing. A telemetry configuration for wound monitoring is depicted in FIG. 4A. The blood sensor 21 can be made part of a surgical dressing or other wound applique. The transponder may or may not be included in this dressing. Operation of blood sensor 21, transponder 23, interrogator 25, and optional annunciator 27 are as described above concerning FIG. 2A. An additional function of data relay means 31 is provided to send bleed information to other destinations such as a local terminal 33, nurses' station display 35, and data logger 37. In FIG. 4B, the blood sensor 21 is present on the patient's body and is electrically connected to a processor 41 either on the patient's body or in proximity thereto. The processor 41 operates on the sensor data for analysis, signal conditioning, and/or formatting and is connected by either wired or wireless link to data relay means 31. In FIG. 4C, only the blood sensor 21 is present on the patient's body. FIG. 5 shows the use of a beacon 26 and beacon receiver 28 in lieu of transponder 23 and interrogator 25 in FIG. 4A.

In the system configurations of FIG. 1 the transceiver 1 may use power from a disposable battery. In the system configurations of FIGS. 2 and 3, the transponder 23 may or may not use battery power (depending on whether the transponder is passive, semi-passive, or active) and the blood sensor and transponder can be implemented as disposable components. If batteries are used for the transponder in application to menstrual sensing, they also would be disposable.

### Physical Implementations

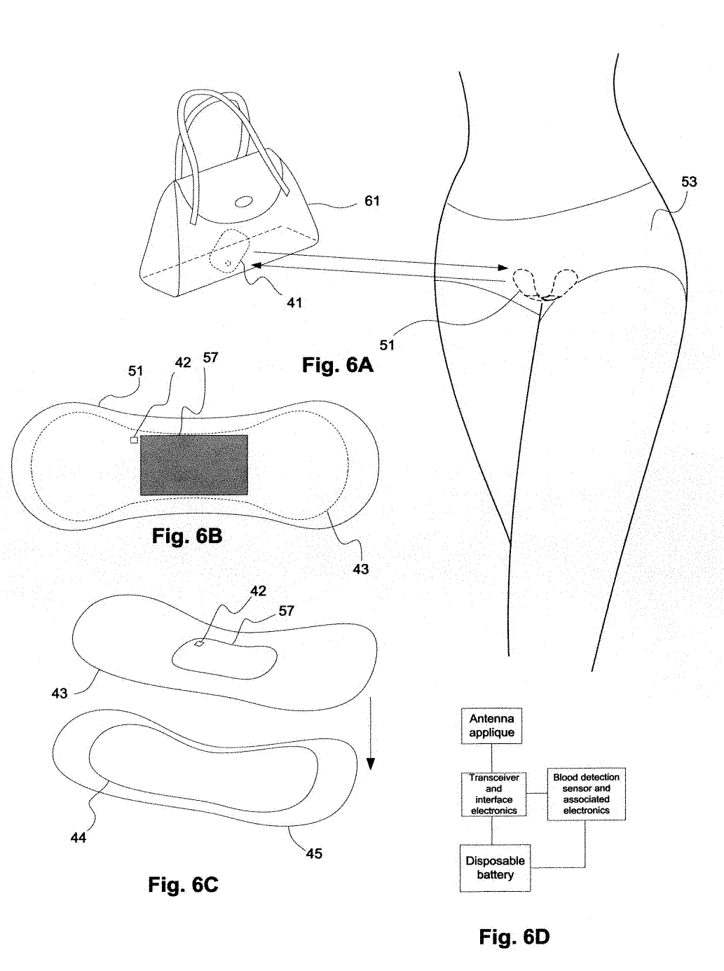

Reference is made to FIG. 6A which depicts use of the disclosed system for the menstrual application in accordance with the system architecture of FIG. 1A. The thin flexible combination of blood sensor and transceiver is disposable, in the form of a pantyliner insert 51, and may be adhesively attached to the undergarment 53. A transceiver chip in the pantyliner communicates periodically with remote transceiver 41, shown carried in a user's purse 61. Upon sensing of blood, a detection signal is provided by the blood sensor 57 to the transceiver 42 which in turn communicates this detection to remote transceiver 41 which causes an audible alert to be issued, such as a ring tone through some form of annunciator.

Reference is made to FIG. 6B which depicts the detail of the pantyliner insert of FIG. 6A. The thin flexible combination of blood sensor 57 with optional interface electronics, transceiver chip 42, thin fabric or paper substrate 43, flexible battery 44, and antenna applique 45 can be made in the form of a wholly disposable pantyliner insert 51, and may be adhesively attached to the undergarment 53.

An exploded pictorial diagram of a candidate construction approach for the pantyliner insert of FIG. 6B is provided in FIG. 6C. On the inner surface of the insert, closest to the body, is the blood sensor 57 that is made part of the thin fabric substrate 43. The next layer can be a thin paper battery 44 and the outer layer can be the body-mountable antenna applique 45. In alternative embodiments of this device, temporary reuse of various system components such as the antenna 45, transceiver 42 and potentially the battery 44 would be permitted by having these components on an applique separate from the blood sensor. Such a separate applique could be attachable to skin or garment with a short, unobtrusive, disposable connection (such as conductors printed on fabric or paper) to a blood sensor pantyliner insert. The motivation for doing this might be cost, given the daily need to replace the pantyliner insert. The non-sensor portion of the system could be reused for the anticipated episode of pre-menstrual blood. The electrical connectivity of the components of FIG. 6C is provided in FIG. 6D.

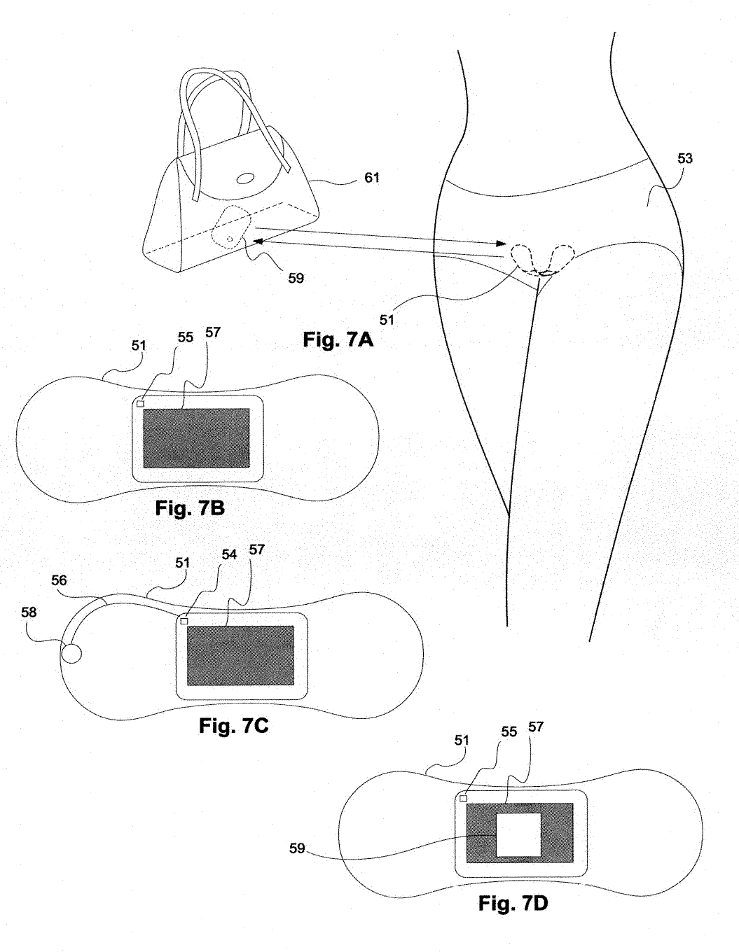

Reference is made to FIG. 7A which depicts use of the disclosed system for the menstrual application in accordance with the system architecture of FIG. 2A. The thin flexible combination of blood sensor and transponder is disposable, in the form of a pantyliner insert 51, and may be adhesively attached to the undergarment 53. It responds to an interrogation by an interrogator 59, shown carried in a user's purse 61. Upon sensing of blood, a detection signal provided by the transponder electronics 55 causes the interrogator module to issue an audible alert, such as a ring tone through some form of annunciator.

In FIG. 7B, a plan view of the insert 51 is provided. The area blood sensor 57 is electrically connected to a radio frequency transponder 55 that returns a blood-detection signal upon query by a radio frequency interrogator module 59. FIG. 7C depicts insert 51 which makes use of a vibratory transducer 58 shown with an electrical connection 56 to power or actuation electronics 54 contained in insert 51; no RF transponder is present in this version of the insert 51. Upon blood detection, the transducer 58 is caused to vibrate to alert the user of need for a menstrual pad. Vibratory transducers (referred to as haptic transducers, these may take the form of eccentric rotating mass motors, linear resonant actuators, or very inexpensive small piezo transducers). FIG. 7D depicts the presence of a LOC type blood analysis sensor 59 that provides information to transponder electronics 55.

The worst case menstrual flow is about 540 milliliters of blood produced in the 5 day average period. This amounts to 4.5 milliliters of flow per hour. The average menstrual pad can absorb about twice this much per hour. To conserve battery life, the transponder is queried periodically rather than continuously. Based on worst case flow rates, a reasonable blood detection query rate is once every few minutes. The transponder 55 may be in the form of an integrated circuit transceiver that receives its operating power from the interrogation energy or it can be a passive device such as a harmonic generator or surface acoustic wave transducer, as will be discussed below. The blood sensor 57 exhibits an alteration in an electrical property such as impedance when blood comes into contact with it. This alteration of electrical property modifies the RF returned by the transponder 55. Detection of this change by the interrogator 59 prompts actuation of the annunciating transducer which alerts the user to the existence of blood.

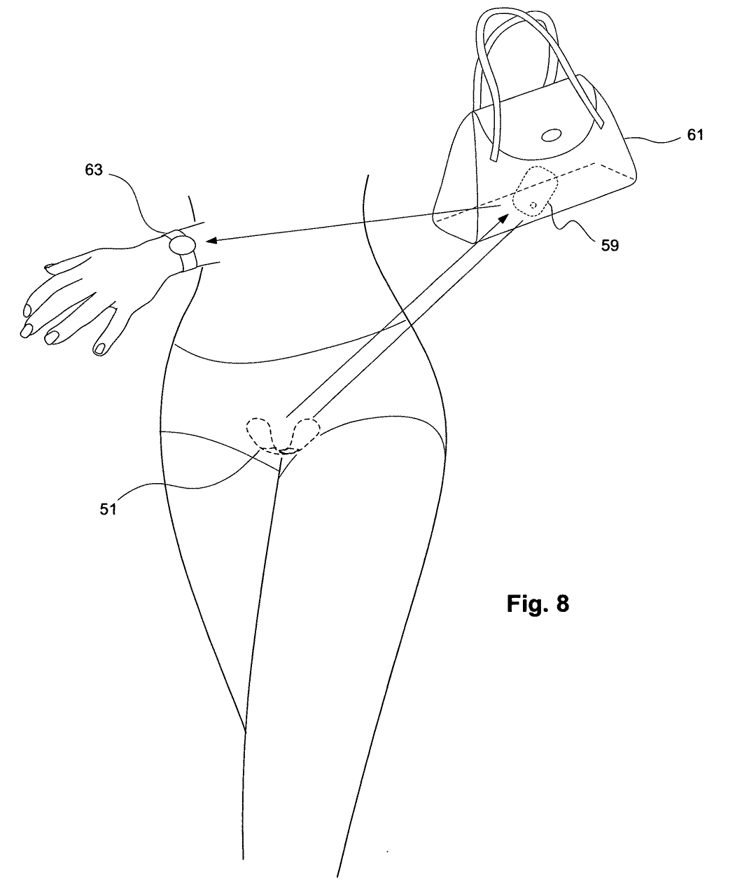

An implementation corresponding to FIG. 2B is shown in FIG. 8. In this implementation, the sensor and transponder insert 51 provides a blood detection response to a query from the interrogator 59. The interrogator 59 then sends a signal to a body worn receiver and vibrating annunciator, shown in FIG. 6 in the form of a wristband 63. The receiver with annunciator can take other forms such as a body-worn patch or other adornment that comes in contact with the body. The receiver could be made part of, or attachable to, a belt or other fashion accessory, especially if in close proximity to the sensor.

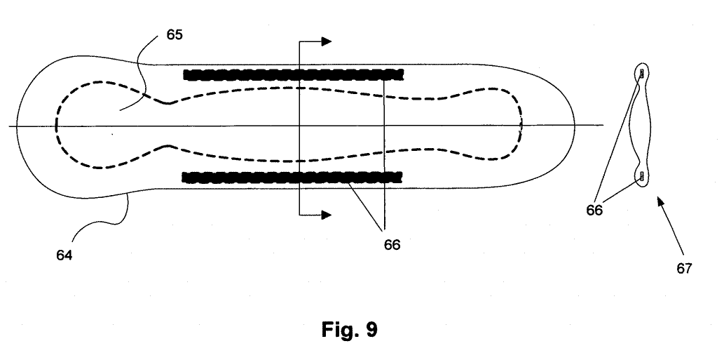

In addition to the uncertainty concerning menstrual onset, there is concern about potential blood leakage from a blood-saturated pad. In another embodiment of the present invention, the configuration of the blood sensor is adapted to detect the spread of blood to the perimeter of the hygiene pad within the thickness of the absorbent material within the pad. FIG. 9 depicts a conventional hygiene pad 64 having a central absorbing area 65. In this embodiment, blood sensors 66 are shown placed in the periphery of the pad 64. The cross sectional view 67 shows the sensors 66 placed internal to the pad, however the sensors could be placed on the inner or outer surface as well. Additionally, various sensor placements could provide an indication of pad saturation on a scale, from 1 to 4, for example. The transponder circuitry and components, not shown, are surface mounted in a convenient location on the pad that does not make skin contact.

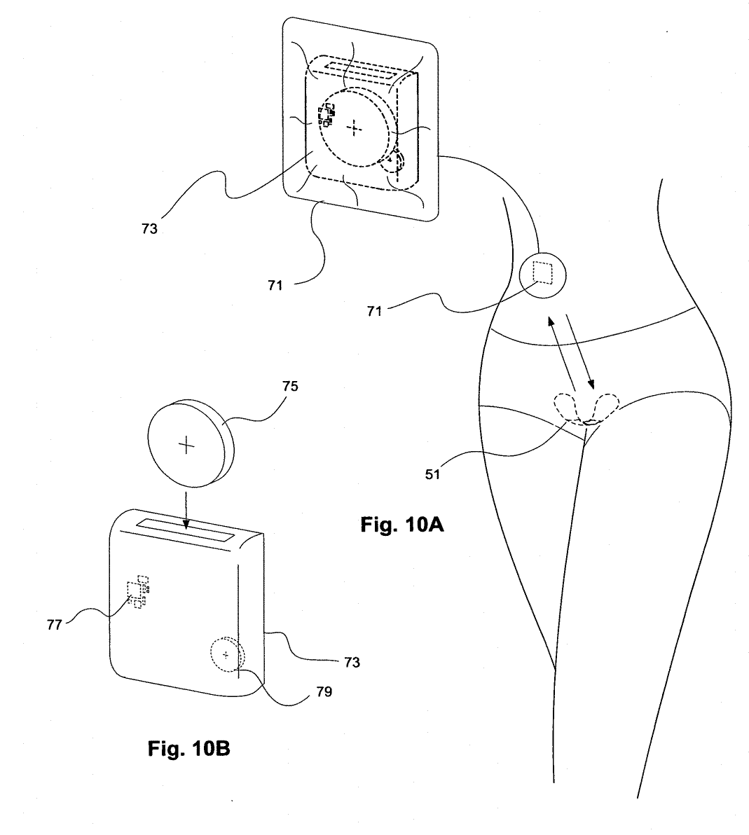

If the RF propagation loss along the body can be overcome by appropriate signaling and processing gain in the system electronics, then the configuration of FIGS. 10A and 10B, can be adopted. In this configuration, the transponder and sensor insert 51 communicates with a body-worn interrogator and annunciator module 73 shown contained in a skin patch 71 (no more than a couple square inches in lateral extent and perhaps ¼ inch in thickness) that is adhesively attached to the skin under clothing, not unlike a nicotine patch or glucose monitoring sensor patch. Other methods of placing the device 73 in contact with the skin are feasible. The body-worn interrogator and annunciator module 73 contains a replaceable battery, which may or may not be rechargeable, the transponder electronics 77, and an annunciator 79 in the form of a vibrating transducer.

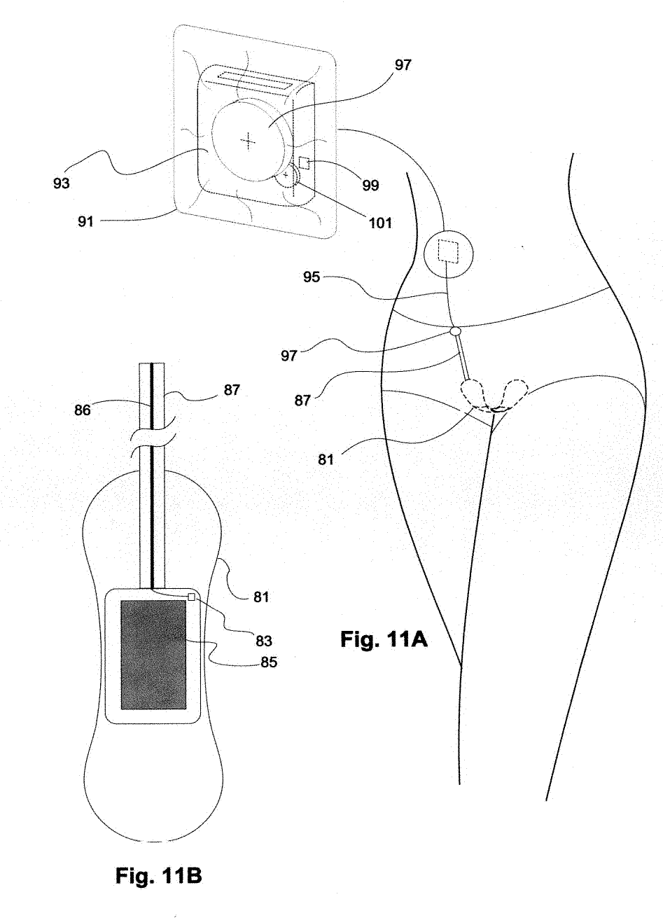

The simple architecture of FIG. 2D is shown implemented in FIGS. 11A and 11B. Herein, the blood detection sensor 85 is incorporated in the pantyliner insert 81. An electrical connection to a body-worn annunciator module 93 is made by a contact strip 87 containing an electrical connection 86 which makes contact with a wire 95 from module 93 through a simple connector 98. The body worn module 93 is shown attached to the abdomen with an adhesive patch 91.

Contained in module 93 are a battery 97, electronics 99 for converting the blood detection signal into a signal to drive the annunciating transducer, and the annunciating transducer 101.

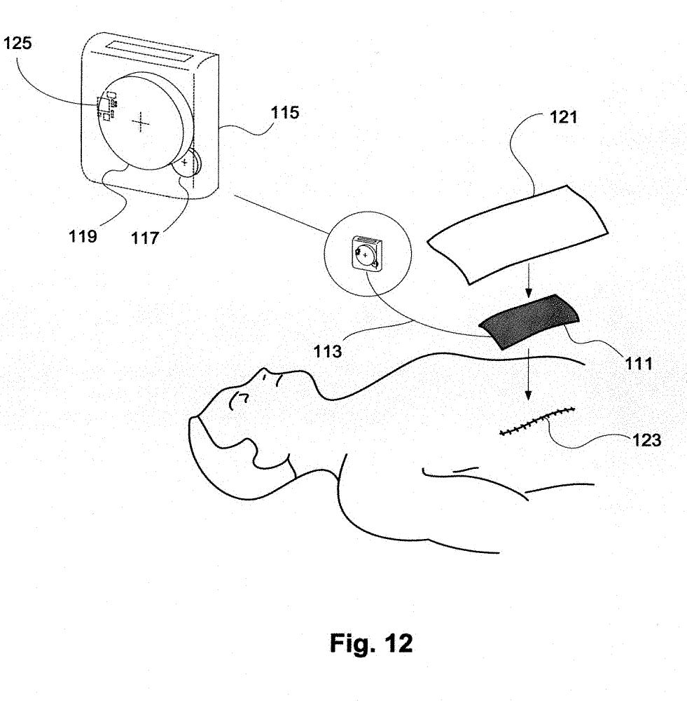

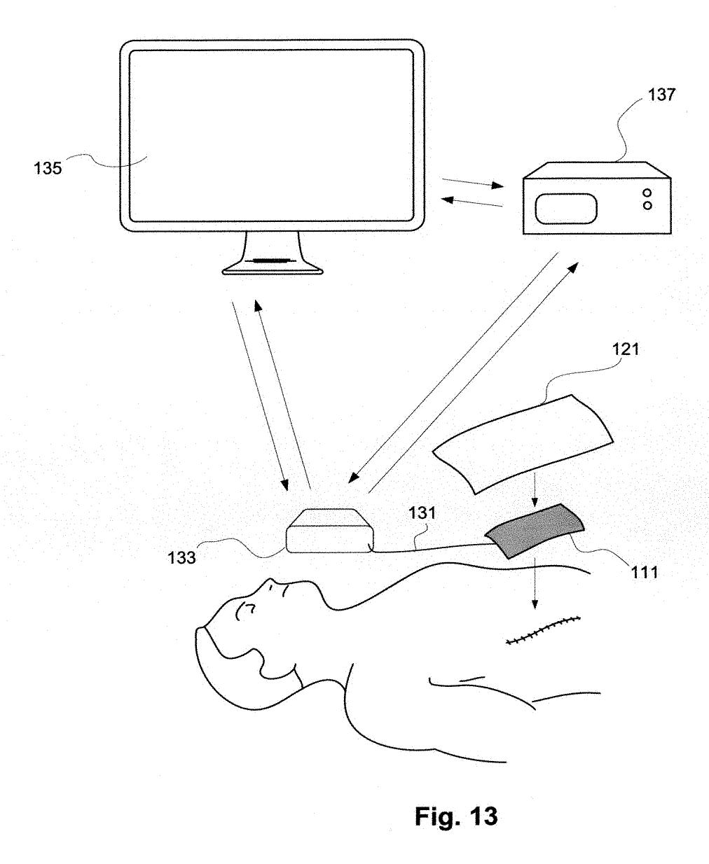

This same configuration can be used for wound monitoring as shown in FIG. 12. The blood sensor 111 is made part of a wound dressing 121 placed in proximity to the sutured wound 123. The blood sensor 111 has an electrical connection 113 to a battery-powered annunciator 115 which may be audible or vibrating, if in contact with the skin. The annunciator 115 contains a battery 119, an annunciating transducer 117 (audible or vibrating), and minimal electronics 125 for converting the blood detection signal into a signal to drive the annunciating transducer 117, and an annunciating transducer 117. In lieu of a battery, an external power supply may be used. An elaboration on the wound monitoring that corresponds to the system of FIG. 4C is depicted in FIG. 13. Blood sensor 111 is electrically connected to processor 133 through connection 131. The processor 133 includes data relay means sending blood detection information to a nurses' station display 135 or recording terminal 137 by RF or infrared communication means. Implementations for monitoring blood loss from dialysis bloodline leakage or other medical intervention that might incur intravenous leakage are within the scope of the present disclosure. In such instances, the blood sensor is configured to conform with an intravenous needle site or at connection locations along the associated bloodline that might leak. Also, the system can be configured to provide a response indicating normal system operation, presence of blood, or absence of blood.

### Blood Sensors

There are alternative methods to sense the presence of blood in real-time. However, from a practical standpoint, only those methods that can be used to alert the user publicly, yet covertly, in a timely manner and without user intervention are within the scope of this invention as applies to menstrual sensing. This comprises physical mechanisms, chemical, or biochemical marker detection schemes that can be transduced into electrical signatures (by impedimetric, amperometric, potentiometric, or optical techniques) that are used to alert the user through various annunciation techniques. Further, these detection schemes must exhibit low false alarm rates for actual blood detection; this rules out the use of moisture detection alone (for the application to menstrual sensing), and detection of components of blood that are common to other bodily and vaginal secretions. A significant challenge is discrimination of menstrual blood from vaginal secretions because compounds such as glucose are found in both. Depending on relative concentrations of such compounds and the ability to normalize such concentrations for adequate threshold detection, glucose may or may not be a useful marker. A determination has been made of 385 proteins that are unique to menstrual blood, not found in circulating blood and vaginal secretions (H. Yan., B. Zhou, M. Prinz, and D. Siegel, “Proteomic analysis of menstrual blood,” Mol Cell Proteomics. 2012 October; 11 (10):1024-35. Epub 2012 Jul 20.)

One or more of these proteins can serve as markers for menstrual blood detection. Consideration must be given to the presence of markers in vaginal secretions that may be present in disease states such as infections, albumin being one such indicator. Nevertheless, candidate markers may include, but are not limited to, albumin, hemoglobin, immunoglobulins, globulins, plasmin, heme, ferritin, transferrin, glucose, A1C, fibrinogen, cholesterol, cortisol, and hormones. Blood albumin and hemoglobin are leading protein markers of choice, given their high blood serum concentration and limited presence in urine and other body fluids. For wound bleed detection, it is possible to use a simple wetness sensor to detect blood emanating from surgical incisions. Such sensors simply detect a conductivity change between electrodes in various geometries that span the detection area of interest. In fact, an area wetness sensor can be used to detect menstrual blood if it is used with another sensor that can discriminate among blood, vaginal fluids, perspiration, urine, and water (in the case of water or beverage spillage) even if this second sensor detects over a small area. One such candidate sensor would be a viscosity sensor fabricated using microfluidic paper sensing as discussed below.

It is anticipated that chemical or antibody-based detection of blood markers can be used in the present invention if these detectors are coupled with approaches to transduce detection as electrical signals. As an example of conventional methods for such transduction, antibody or other chemical binding methods have been used to transduce extremely small marker concentrations using surface acoustic wave (SAW)-based oscillators. Antibodies to the markers in question are attached to the SAW substrate, as the antibodies bind the marker molecules, the SAW oscillator frequency is shifted in a marker molecule concentration-dependent manner based on mass effects (Stubbs, D. D. , Sang-Hun Lee, and Hunt, W. D., “Molecular Recognition for Electronic Noses Using Surface Acoustic Wave Immunoassay Sensors,” IEEE Sensors Journal, vol. 2, no. 4, pp. 294-300, 2002). This approach is useful for vapor detection or for marker-containing liquids that are washed from the sensor with subsequent sensor drying. Shear wave SAW sensors used for detecting markers in liquids that maintain continuous contact with the sensor require thick substrates inappropriate for the presently-disclosed application.

Additionally, there are ways to employ SAW technology for blood sensing. These approaches can operate in the continuous presence of liquid blood because they do not rely on mass effects and do not involve direct fluid contact with the SAW device that would influence its response. Rather, such approaches involve combining SAW transducers with blood sensors that make contact with liquid blood. The CNT-based sensor described below, would be a good candidate for sensors of this kind that vary critical performance parameters of the SAW device such as surface wave propagation velocity by electro-physical means.

### Chemiresistors and Impedimetric Sensors

There is a method for transduction of chemical detection to electrical signal that is particularly cost-effective and application appropriate. It involves immobilization of molecules having affinity for the target marker on carbon nanotube (CNT) substrates. The resulting activated CNTs exhibit a marker concentration-dependent electrical conductivity.

In a first example, a research group at the University of Michigan has impregnated cotton fibers with CNTs having attached anti-blood albumin antibodies. The resulting cloth exhibits dramatic, highly-specific conductivity increase upon exposure to human blood albumin. (Shim, B. S., Chen, W., Doty, C., Xu, C. L., & Kotov, N. A. (2008). Smart Electronic Yarns and Wearable Fabrics for Human Biomonitoring made by Carbon Nanotube Coating with Polyelectrolytes. Nano Letters, 8 (12), 4151-4157.). One of the issues associated with use of antibodies for assay of chemical markers is the fragility of antibody molecules. For adequate shelf life, antibodies typically are stored at low temperatures or subjected to lyophilization. Commercial stabilizers are available to optimize shelf life. For example, Stabilguard biomolecule stabilizer (SurModics Inc.; Eden Prairie, Minn.) and Stabilcoat immunoassay stabilizer (SurModics Inc.) have been shown to preserve activity of dried monoclonal and polyclonal antibodies for more than 18 months at room temperature.

In more recent work at Youngstown State University (P. Cortes, A. Olszewski, and D. Fagan, “Blood Detection Using Biological Modified CNTs,” American Institute of Chemical Engineers, 2013 Annual Meeting, Materials Engineering and Sciences Division.), a short chain peptide, GAQGHTVEK (GK-1) has been shown to bind specifically to serum albumin preferentially over bodily fluids. GK-1 was covalently attached to the surface of carboxylated multi-wall CNTs (MWCNTs). Common synthetic threads were coated by the biological modified MWCNTs through a dipping process, resulting in a semi-conductive bio-sensing thread. When exposed to serum albumin, the threads exhibited significant increase in resistance in contrast to the reduction in resistance cause by saline and other solutions. A thread of length less than half inch exhibited a change from 100 ohms to 230 ohms upon exposure to glucose at the normal concentration found in human blood. Work is contemplated in the CNT design and peptide attachment approach that will significantly increase this resistance change. Additionally, a folded, reticulated fiber of long effective length would provide a significantly greater change in terminal resistance especially when the entire length is exposed to blood glucose. This is a preferred type of blood sensor for the present invention given a high marker specificity combined with immediate variation in sensible conductivity, the temperature stability of the albumin binder, and the feasibility of a large sensing area achievable in an activated CNT fabric using this approach. Use of anticoagulants embedded in the sensor or insert and uptake of blood into the sensor volume by capillary and other action is anticipated.

The configuration of the blood sensor in an absorbent pad or fabric will depend on the nature of the detection phenomenology. In the case of a chemiresistor, if the detection results in a decrease in impedance, then an area of chemiresistors connected in parallel electrically is an appropriate configuration; this registers a blood-induced electrical “short”. Conversely, if the detection results in an increase in impedance, then an area of chemiresistors connected in series, electrically, is appropriate, thereby registering a blood-induced electrical “open” condition.

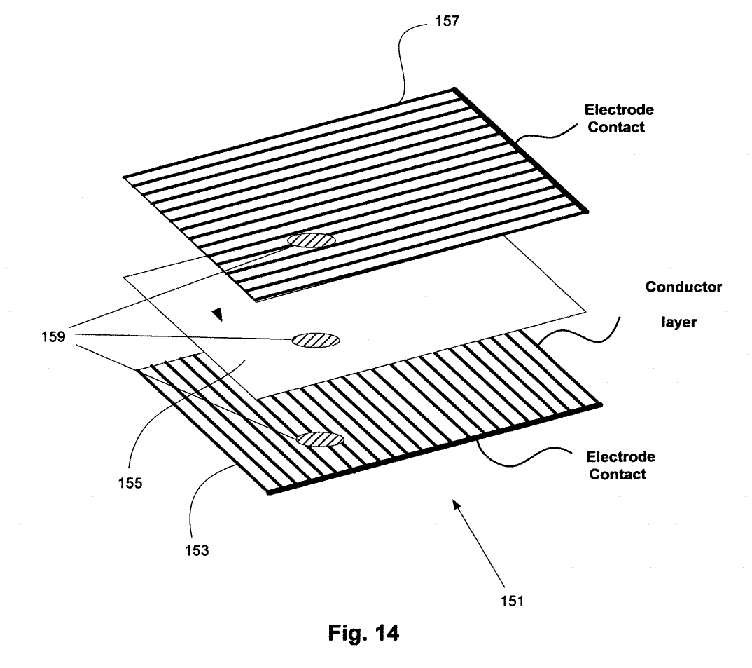

The initial presence of menstrual blood is confined to the central area of an undergarment. However, the sensor of the present invention must detect the presence of a single droplet of blood anywhere within an approximately 1.5 inch by 2 inch rectangular area. A straightforward approach to achieving a conductivity indication over such an area is to permit the modification of the electrical impedance between two conductor planes by the presence of blood as shown in FIG. 14. The top fabric layer 157 and bottom fabric layer 153 of the sensor 151 have impregnated conductors. The middle fabric layer 155 is impregnated with CNT functionalized for affinity to a particular blood marker material and which reduces impedance in the presence of said marker. When a drop of blood 159 is introduced to the top layer, it quickly diffuses into the middle layer and establishes a high conductivity connection between the upper and lower layers, thereby providing a low terminal sensor impedance or “short” condition. As stated, this thin, cloth-based sensor can be adhesively applied directly to the undergarment or to a light weight menstrual pad. Further, the CNT irreversibly adheres to cotton and will not provide any biocompatibility issues. Also, the use of paper in lieu of fabric is within the scope of this disclosed concept. This sensor is electrically connected to the transponder circuitry as described below.

The sensor of FIG. 14 is a model for other impedimetric sensors that result in an impedance change upon exposure to the target molecule(s) and can be woven into fabrics, used to impregnate them. These include nanosensing approaches such as nanowire electrodes for markers like glucose (M. Zhang, F. Cheng, Z. Cai, and H. Yao, “Glucose Biosensor Based on Highly Dispersed Au Nanoparticles Supported on Palladium Nanowire Arrays,” Int. J. Electrochem. Sci., No. 5 (2010) pp. 1026-1031). (M. Swierczewska, G. Liva, S. Leea, and X. Chena, “High-Sensitivity Nanosensors for Biomarker Detection,” Chem Soc Rev. 2012 Apr. 7; 41 (7): 2641-2655.).

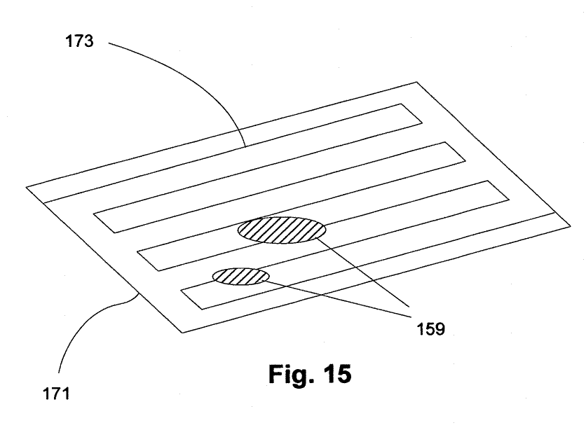

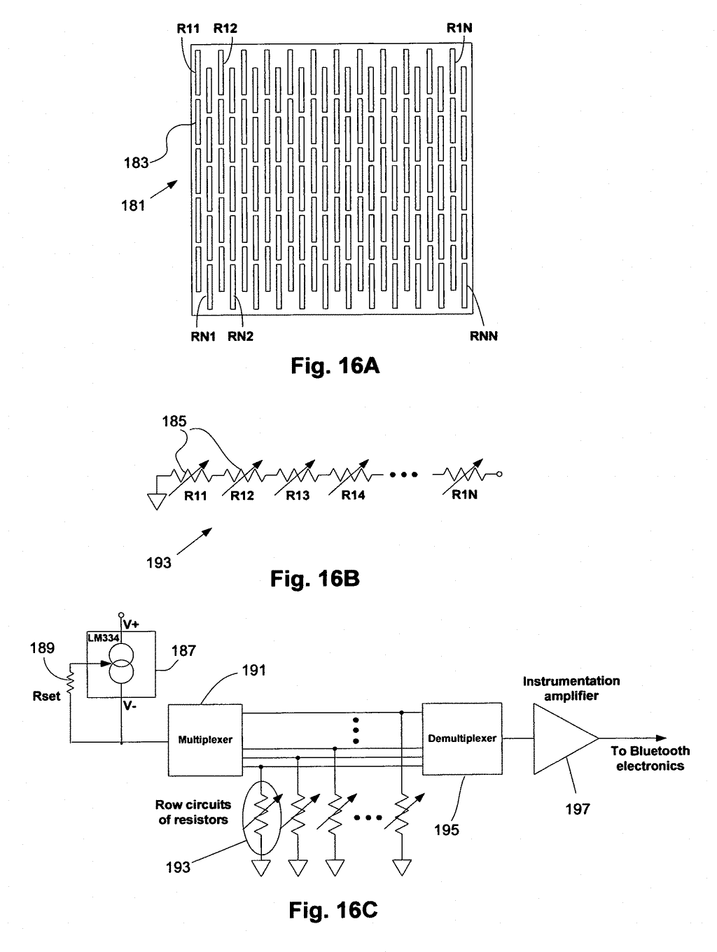

The sensor configuration of FIG. 15 corresponds to an impedimetric sensor 171 that increases impedance upon exposure to blood. The terminal impedance will monotonically increase with blood deposits 159 along the chemiresistor conductor 173. FIGS. 16A, B, and C elaborate on this concept exploiting a graphene impregnated paper or fabric. The work of Heldt et al. (C. L. Heldt, A. K. Sieloff, J. P. Merillat, A. R. Minerick, J. A. King, W. F. Perger, H. Fukushima, J. Narendra, “Stacked Graphene Nanoplatelet Paper Sensor for Protein Detection”, Sensors and Actuators B, Vol. 181, pp. 92-98, May 2013.) has demonstrated a graphene impregnated paper with a change in resistivity of 700 ohms per square upon exposure to albumin concentration found in human blood (approximately 600 micromolar). The baseline wet resistivity of this material is close to 100 ohms per square. FIG. 16A is a depiction of a wide area blood sensor 181 extending perhaps a few inches on each side. Some number of strips of this graphene impregnated paper or cloth act as blood sensing variable resistors 183 and are shown distributed in a two-dimensional pattern to span the area where blood could be deposited. It is possible to diminish the number of such resistors required by either placing fabric-borne resistors of this kind in contact with a superhydrophilic surface (as discussed below) or printing the resistor sensors and electrical connections directly on a superhydrophilic surface. The superhydrophilic surface will distribute a single drop of blood over a wide area to make fluid contact with a given resistor.

Each variable resistor 185 of FIG. 16B represents a certain length strip of such graphene impregnated paper or fabric. In fact, graphene-based inks can be used to print resistor patterns onto fabric substrates and highly conductive conventional carbon-based inks can be used to make interconnects in these patterns. The length-to-width ratio of these strips multiplied by the ohms per square resistivity of this material at its given resistive state yields the resistance of such a discrete resistor. A row of such resistors is depicted as electrically connected in series to create a row of connected sensing resistors. If a drop of blood wets any one of the individual resistors, that resistor's resistance will be increased by a factor of 8. Hence, if, for example, 8 or so resistors in a row are serially connected and blood wets any one of the individual resistors, the terminal resistance of this series circuit will almost double. By injecting a constant current into this series circuit, a sensible voltage change will occur. It is possible to connect more than just 8 resistors in series to increase the spatial coverage of blood detection by a single serial connection of resistors by making trades offs among a) the sensing material composition and sensitivity to blood markers, b) the form factors of individual resistors, c) individual resistor lengths, d) the magnitude of injected current and e) signal conditioning efforts. Ideally, all the resistors spanning the blood sensor footprint would be connected so that blood detection would require threshold detection of a single voltage across this circuit. However, if this is not feasible for signal-to-noise reasons, the voltages across a limited number of such resistor circuits can be multiplexed for blood detection. FIG. 16C depicts the injection of current from a constant current source 187, such as a Linear Technology LM334 IC, into an analog multiplexer (MUX) 191 which sequentially excites each row circuit of resistors 193. The analog demultiplexer (DEMUX) 195, synchronous with the multiplexer, samples the voltages across each row circuit of resistors (Various analog MUX/DEMUX parts are commercially available to achieve this functionality such as a CMOS SN74HC4852). These voltages are amplified by an instrumentation amplifier 195 for subsequent threshold detection of blood drop presence by a voltage comparator or analog-to-digital converter that would be part of a Bluetooth sensor transceiver or similar electronics. This is sensor implementation is exemplary of using materials that increase their impedance in the presence of blood. In the case of sensing materials that exhibit impedance decrease with blood presence, sensing resistors made from such material that span the sensing area can be placed electrically in parallel, given that net circuit resistance will be governed by the least resistance element (the blood-exposed element).

### Amperometric Sensors

Amperometric sensors provide a terminal current upon detection of the marker of interest. The most common amperometric sensor technology is used for diabetic monitoring and is well developed, with numerous handheld glucose meters available that use blood test strips. An analogous disposable hemoglobin test strip is disclosed in U.S. patent application with publication number U.S. 20090321254A1.

Glucose sensing devices typically exploit a two-step chemical process involving test strips containing reagents. An example chemistry uses glucose dehydrogenase to convert glucose in the blood sample to gluconolactone. This reaction liberates an electron that reduces hexacyanoferrate (III) to hexacyanoferrate (II). A voltage is applied between two identical electrodes spanning the blood sample, which reoxidizes the hexacyanoferrate (II). This generates an electron flow proportional to the glucose concentration of the sample. The current is in the 10's of microamperes range and is converted to a sensible voltage by means of a transimpedance amplifier. It is feasible to create sets of interdigitated electrodes of the kind used for glucose sensing in order to provide blood detection over an area of a few square inches. Also, such a sensor can be operated at zero bias to simplify the system design. Detection of glucose for the indication of blood may require some thresholding assessment.

### Potentiometric Sensors

Potentiometric sensors comprise chemical sensors that measure an electrode voltage upon detection of a chemical marker of interest. Among this category of sensors, are field effect transistor (FET)-type biosensors that exploit detection of ion exchange at the FET gate. The species to be detected and the sensor's selectivity to those species can be determined by the materials coated on the surface of the gate insulator. Ion sensors, biosensors, and oxygen sensors have been developed using polymer membranes, immobilized enzyme membranes, and solid electrolytic films. (Principles of Bacterial Detection: Biosensors, Recognition Receptors and Microsystems, Edited by M. Zourob, S. Elwary, A. P. F. Turner, Springer, 2008.)

Nanowire chemical sensors comprise another technology that also can be utilized for potentiometric sensors in the present disclosure. They have attracted much attention for two reasons. First, their large surface area to volume ratio promises high sensitivity. Second, the size of the nanostructures is similar to the size of species being sensed, thus the nanostructures make good candidate transducers for producing the signals that are then read and recorded by conventional instruments. The underlying phenomenon exploited in using nanowires is the field effect on which field effect transistors (FETs) are based (R. M. Penner, “Chemical sensing with nanowires,” Annu Rev Anal Chem (Palo Alto Calif.). 2012; 5:461-85. doi: 10.1146/annurev-anchem-062011-143007. Epub 2012 Apr. 9.).

### Optical Sensors

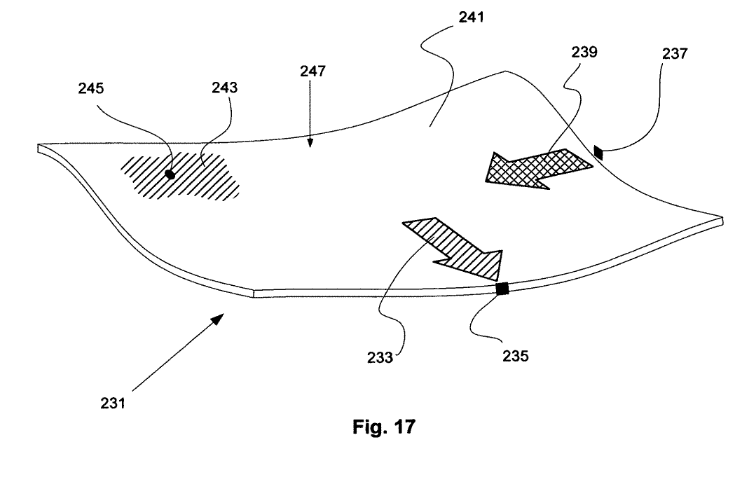

FIG. 17 depicts a large area blood detection sensor 231 based on an optical approach. This type of approach is analogous to a luminescent solar collector, well known in the prior art, which collects ultraviolet (UV) light in a UV-transparent structure that downconverts the UV to visible light which then is captured by total internal reflection for detection. Herein, an optically clear flexible substrate such as a high grade silicone rubber sheet 241 is provided as a deposition surface for blood droplets. A light emitting diode (LED) or low power laser diode 237 is depicted as introducing light, represented by arrow 239 into sheet 241 along one edge of the sheet. Micro LEDs exhibiting current drains in the microamps range are ideal for this application. One or more such LEDs can be used for pulsed excitation light. The timing of LED pulsing can be controlled with electronics to coordinate with the polling of the blood sensor and periodic communication of the sensor output by a radio frequency communicator. Various means of efficiently coupling this light into sheet 241 are envisioned including, but not limited to, a planar tapered waveguide, molded lenslets, etc. The thin film sheet 241 serves to conduct this light throughout its volume by total internal reflection and scattering. The upper surface 247 of sheet 241 can be made hydrophilic even if the polymer composing the sheet is hydrophobic as per the discussion below. This can be done by texturing or chemically processing the surface. This causes an initially deposited blood droplet 245 to spread over an area 243 of the surface for more efficient exposure to excitation light. If the surface 247 is treated with appropriate reagents such as a fluorogen and glucose oxidase, for example, then area 243 will be caused to fluoresce as it is excited by the light from LED (or laser diode) 237 coupled into the blood-reagent mixture from the sheet 241. Light will be preferentially out-coupled from the sheet 241 into the blood reagent mixture given that the index of refraction of blood serum is closer to that of the polymer than surrounding air (the blood serum acting as an index-matching fluid). Conversely, light, represented by arrow 233, from the fluorescing area 243 will be coupled back into sheet 241 and conducted to a photodetector 235 shown mounted proximate to another edge of the sheet 241. Lateral placement of the photodetector 235 along an edge of sheet 241 will not be critical, given the efficiency of light conduction by the sheet.

However, various strategies are considered by which fluorescent light collection may be optimized; these include modifications to the surface of sheet 241 for waveguiding, the use of waveguide conduits for conducting light from the various edges of sheet 241 to the single photodetector, etc. The fluorescing reagents and LED wavelength are chosen so that the blood-reagent mixture is illuminated by excitation energy over a range of wavelengths that are sufficiently separated from the wavelength range of the fluorescence. This permits blocking of all illuminating light from being detected by the photodetector 235 through use of a short wavelength optical filter or a photodector 235 with only longer wavelength sensitivity. This blood detection approach affords sensitivity to deposition of a single blood droplet anywhere on the surface of sheet 241. Additionally, sheet 241 can be highly perforated so that it provides a “breathable” layer of material in a pantyliner insert. Also, it can be made to disperse a single drop of blood over a large area by texturing of the surface to create extreme hydrophilic behavior, as discussed below.

It is also considered that an optical sensor can detect blood based on its passive spectral characteristics. This can be done in the visible and/or infrared spectrum. A micro white light (or infrared) LED illuminator can be used in concert with a set of spectral detectors at various characterizing wavelengths to monitor reflection or absorption blood spectra. Another approach is to use a specific wavelength illuminator with corresponding wavelength detector for detection at a specific wavelength band. For this purpose, devices such as the surface mount red, blue, green color light sensor chips are available from Everlight Electronics Co., Ltd. Taipei, Taiwan. The CLS15-22C/L213/TR8 series devices comprise one channel Si photodiode sensitivity to the red, green or blue region spectrum in a miniature SMD package. Alternatively, the red, green, and blue channels can be combined in a single chip such as the TCS3103 Color Sensor, from AMS AG of Austria. This high sensitivity device is provided in 2 millimeter square package and can be used for spectral determination of blood presence when used with appropriate low power LED illumination. The physical illumination of blood and detection of associated light absorption or reflection can be achieved with light coupled through clear polymer substrates as used in the fluorogenic approach. Receptacles for blood can be made in optical channels that carry the illumination light. Blood can be transported from a large area by capillary/fluidic means in order to deposit a blood sample in a small sensing receptacle by microfluidic means or textured surfaces. Recently, Spectra-Physics, the laser manufacturer, has developed a method of fabricating superhydrophobic and superhydrophilic surfaces for biomicrofluidics applications by femtosecond laser processing of polymer or glass surfaces. Inherently hydrophobic surfaces can be made superhydrophilic by appropriate micro-texturing of the surface. Geometries supporting measurement of reflected light as well as transmitted light to measure absorption are well-developed in the prior art (E. Carregal-Romero, B. Ibarlucea, S. Demming, S. Büttgenbach, C. Fernández-Sánchez, and A. Llobera, “Integrated Polymeric Light Emitter for Disposable Photonic Lab on Chip Systems,” 16^{th} International Conference on Miniaturized Systems for Chemistry and Life Sciences, Oct. 28-Nov. 1, 2012).

### Microfluidic Technology

An emerging class of sensors employs paper-based microfluidic devices (Z. Nie, C. A. Nijhuis, J. Gong, X. Chen, A. Kumachev, A. W. Martinez, M. Narovlyansky. and G. M. Whitesides, “Electrochemical sensing in paper-based microfluidic devices,” Lab Chip, 2010, 10, 477-483.) These sensors exploit microfluidic channels, fabricated from patterned paper (typically, either chromatography paper or a polyester-cellulose blend) with sensing electrodes printed in proximity to these channels. Hydrophobic barriers are created in the paper by wax or polymer patterning on the paper in order to confine liquids in the microchannels. The wicking behavior of these channels can be used to collect and transport the fluid(s) of interest, such as blood, for sensible testing. Various geometries can be used to collect fluid from across large relatively large areas, so that the number of sensor points can be reduced. Exemplary of fluid collection technologies that can capture fluids over a large area and direct them to a destination location are microfluidic films as disclosed in U.S. Pat. No. 7,910,790 to Johnston, et al.

A LOC is a device that integrates one or several laboratory functions on a single chip of only millimeters to a few square centimeters in size. LOCs deal with the handling of extremely small fluid volumes down to less than pico liters. LOC devices are a subset of MEMS devices. LOC is closely related to, and overlaps with, microfluidics which describes primarily the physics, the manipulation and study of minute amounts of fluids. However, strictly regarded LOC indicates generally the scaling of single or multiple lab processes down to chip-format (Lab on a Chip Technology: Fabrication and microfluidics, Volume 1, edited by K. E. Herold, Avraham Rasooly).

A single marker such as a common bacterial metabolite may be used to indicate the likely presence of odor causing bacteria in a menstrual pad. Also, LOC technology can be used to detect menstrual pad odor or the bacteria causing such odor, providing indication of need to change a menstrual pad.

The so-called LOC technology can be employed to monitor presence and levels of blood constituents and markers as well as skin for health diagnostic purposes and can be fabricated on a paper substrate as fully disposable (G. Chitnis, Z. Ding, C. L. Chang, C. A. Savran, and B. Ziaie, “Laser-treated hydrophobic paper: an inexpensive microfluidic platform, Lab Chip. 2011 Mar. 21; 1 1(6):1161-5. doi: 10.1039/c0lc00512f. Epub 2011 Jan. 24.). This can apply to monitoring of menstrual blood (menstrual embodiment), wound exudates (wound bandage embodiment), and skin surfaces (smart bandage embodiment).

It can be considered that a wetness sensor used for blood detection also could be used for urine detection. This might be especially useful in the present system disclosure for the purpose of alerting a sleeping woman of the incidence of nocturnal urine leakage. The annunciation mechanism would wake her from a sound sleep or alert her caretaker (or nurses' station).

### RF Communication Components

RF components for the presently disclosed system preferentially operate in unlicensed radio bands. Example unlicensed ISM and short range device bands include 315, 433, 915, 2400 MHz (Consideration should be given to potential interference from key fobs, door openers, cell phones, computer networks, wireless speakers and headphones in the 915 MHz band).

Unlicensed operation is permitted in the 60 GHz band because it is subject to heavy attenuation by atmospheric oxygen resonance absorption, facilitating spatial channel reuse. Compact beamforming technology will permit effective point-to-point pencil beam connectivity and retrodirective (phase conjugate) array antennas can permit robust connectivity between moving communication nodes (S. Gupta, “Automatic Analog Beamforming Transceiver for 60 GHz Radios,” E-print archive: arXiv:0901.2771v1 (2009)). Antennas are small at these millimeter wavelengths supporting implementation of the presently-disclosed system. A beam sweep protocol would be initiated for the purpose of receiver antenna acquisition by the transmitting portion of the system.

Various off-the-shelf low power communication chips are commercially available that can be used in a transceiver-based , an interrogator and transponder-based, or a beacon and beacon receiver-based implementation of the disclosed system. These offer programmable selection from among a set of modulation types. Additionally, spread spectrum systems operable in the ISM bands can provide low power links with good link margins that may be necessary in the face of large propagation path losses anticipated for some modes of on-body transmission. All such systems are within the scope of the present invention.

Among the alternatives for transceiver-based implementation, a leading candidate technology is Bluetooth Low Energy as this communication standard offers numerous advantages over other schemes; it has been rendered in highly integrated hardware chip configurations that include functionality to support internet-of-things (IoT) sensor beacons.

For the case of a transponder implementation, to minimize complexity, emphasis is placed on those communication components with signaling strategies that support a passive transponder design. So, in addition to low power communication chips, chirp-based pulse compression, harmonic generation schemes, and passive or semi-passive RFID tags are considered among preferred methodologies. In chirp-based pulse compression, the interrogator transmits a frequency chirp signal and the transponder effectively autocorrelates the chirp to achieve processing gain. In harmonic schemes, the transponder returns a harmonic (typically the 2^{nd} harmonic) of the interrogation signal. RFID tags are a mature technology that abides by sophisticated standards; the sensor tag instantiations are particularly relevant to this disclosure.

A harmonic scheme that enjoys processing gain requires that the modulation type be chosen to avoid mixing products that would result from the nonlinear device that achieves harmonic generation in the transponder. Instantaneous single frequency transmission is associated with frequency hopping and a degenerate form of hopping is binary, frequency shift keying (FSK). A high processing gain system could use simple FSK with a long pseudo-random (PN) keying code. The processing gain is given by

Gₚ=10 log(N_{c})

Where N_{c} is the length of the binary PN code. For example, 60 dB of processing gain would require a code length of 10⁶ bits. The rapid acquisition/detection of such codes is well developed in the prior art and derives from the ranging codes first used in the Deep Space Network. Short preamble or acquisition codes can be made part of the longer code for his purpose. Alternatively, a matched filter may be used for a fixed code, as well known in the prior art, in which case there is no acquisition requirement.

U.S. Pat. No. 8,002,645 to Savarese et al. discloses a system that uses a direct sequence spread spectrum wherein the spreading code is applied to a carrier using binary phase shift keying (BPSK) and dispreading is done in a passive tag by “squaring” of the signal with a diode nonlinearity. This approach can be employed in the present invention with or without other levels of coding.

An exemplary design uses devices operating in an ISM band, specifically, an interrogator transmitter at 2.4 GHz in concert with a receiver operating at the second harmonic, 4.8 GHz. Other fundamental and associated second harmonic frequencies can be used. Commercially-available transceiver chips can be adapted for reception at the second or third harmonic of the transmit carrier frequency. This can be accomplished either by appropriate mixing of the harmonic down to the transmit carrier frequency or by using a receiver at the harmonic frequency.

Among the alternatives for the receiver are various commercially available receiver ICs or RF front ends and IF circuits. Additionally, a custom ASIC can be created for this application. An example low power, high sensitivity 5 GHz CMOS receiver design is provided in “A Fully Integrated CMOS Receiver,” PhD Dissertation by D. Shi, University of Michigan, 2008. A coded PAM modulation scheme and narrowband IF filtering should mitigate interference from other ISM band sources.

A candidate interrogator transmitter is found in the Texas Instruments CC2500. Coded OOK modulation can be used as the interrogation signal. Using a high side injection with a low noise mixer, the second harmonic signal can be received with the receiver in this chip.

### Implementation Using Transceivers

Use of transceivers at each end of the communication link obviously improves the link margin over the use of passive and semi-passive transponders with associated interrogators. As previously mentioned, among transceivers that have widely adopted, highly functional protocols, are those using the Bluetooth Low Energy (BLE) standards.

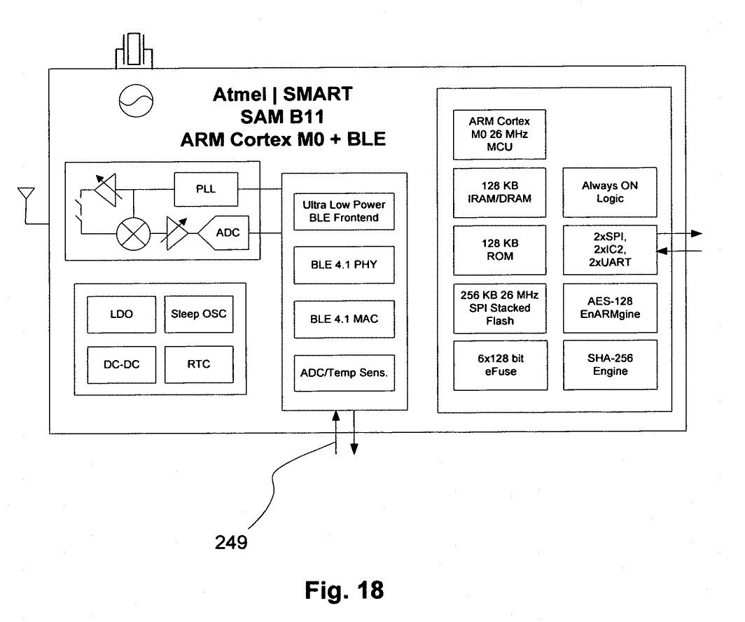

A number of manufacturers offer single chip implementations of BLE transceivers integrated with MCUs. These often include on-chip ADCs for direct interface with analog sensors. A functional block diagram of an exemplary device manufactured by Microchip Atmel is shown in FIG. 18. The chip contains an analog-to-digital converter (ADC) and thereby offers a direct analog sensor interface 249 that can accept, for example, a blood sensor voltage input. The chip implementation of this device measures 2.2 mm by 2.1 mm.

### Interrogator and Transponder Implementation

The transponder may be active (consuming battery power), semi-passive (making use of some portion of the interrogation energy to power a response), or passive (simply returning some portion of the interrogation energy in a modified or unmodified form). All such types of transponders are well known in the RFID tag prior art. Although these three types of transponder are within the scope of the present invention, because of the disposable nature of the device, it is preferable that the transponder respond passively or semi-passively to interrogations. Leading candidate passive transponder technologies include SAW devices used in systems exhibiting processing gain, harmonic generators, and passive and semi-passive RFID sensor tags.

A SAW orthogonal frequency coding (OFC) delay line has been demonstrated to exhibit low loss as a reflector (6-10 dB). A transceiver using a chirp waveform at 915 MHz with an OFC SAW delay line-based tag of this kind has realistically achievable loop gains between 100 and 180 dB. (D. R. Gallagher, D. C. Malocha, D. Puccio, and N. Saldanha, “Orthogonal Frequency Coded Filters for Use in Ultra-Wideband Communication Systems,” IEEE Transactions on Ultrasonics, Ferroelectrics, and Frequency Control, Vol. 55, No. 3, March 2008, pp. 696-703.) Such gain will be important given the path losses associated with on-body communication discussed below. Further, it is possible to render such OFC SAW devices with coatings on disposable flexible plastic films (H. Jin, J. Zhou, X. He, W. Wang, H.i Guo, S. Dong, D. Wang, Y. Xu, J. Geng, J. K. Luo, and W. I. Milne, “Flexible Surface Acoustic Wave Resonators Built on Disposable Plastic Film for Electronics and Lab-On-A-Chip Applications,” Scientific Reports 3, Article number: 2140, Published 5 Jul.2013.).

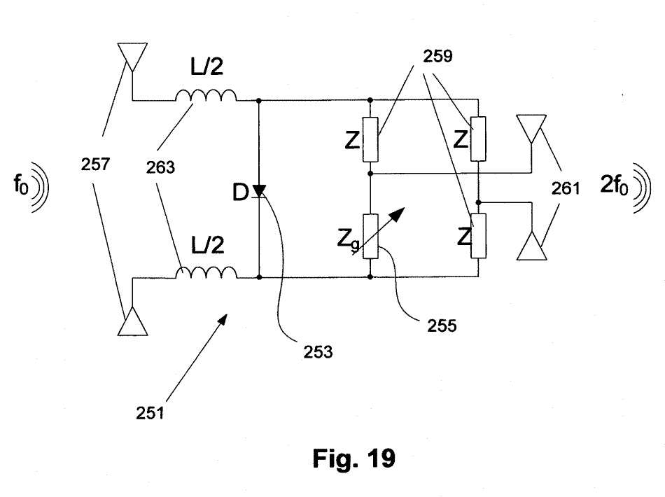

Reference is made to FIG. 19 depicting a harmonic-generating passive transponder 251 which uses a passive nonlinear component such as a varactor 253 so that the interrogation signal occurring at frequency f₀ elicits a frequency-multiplied output signal from the transponder 251. The output signal is to be modified by the blood sensor impedance 255 so as to indicate detection of blood. In an adaptation of the device of Baccarelli et al. (R. Baccarelli, G. Orecchini, F. Alimenti, and L. Roselli, “Feasibility study of a fully organic, CNT based, harmonic RFID gas sensor,” in Proc. IEEE Int. Conf. RFID-Technologies Applications, November 2012, pp. 419-422.) for the present invention, the output signal should be of zero power in the absence of detected blood and be of significant power level in the presence of detected blood. Because the terminal impedance of the sensor, without the presence of blood, at the output frequency 2f₀ exhibits finite resistive and reactive component values, it is necessary to place the sensor in the Wheatstone bridge configuration (comprising impedances 255 and 259) shown in order to approach this ideal response. The impedances 259 should match the value of the sensor impedance 255 when there is no blood detection; this leads to zero output power from the balanced bridge condition. Further, given the variability of the parasitic reactive components of the sensor, a capacitor can be placed in parallel, of value large enough to dominate the reactance yet small enough to permit a significant transponder output detection signal. Inductors 263 are for impedance matching to the receiving antennas 257. The output antenna 261 is optimized for the harmonic frequency. Poor link margins result from using a passive transponder in the presence of body-mounted propagation loss. However, there is the prospect of combining this passive transponder concept with a dual frequency, or broadband antenna design based on the special low loss body-mounted antenna design discussed below.

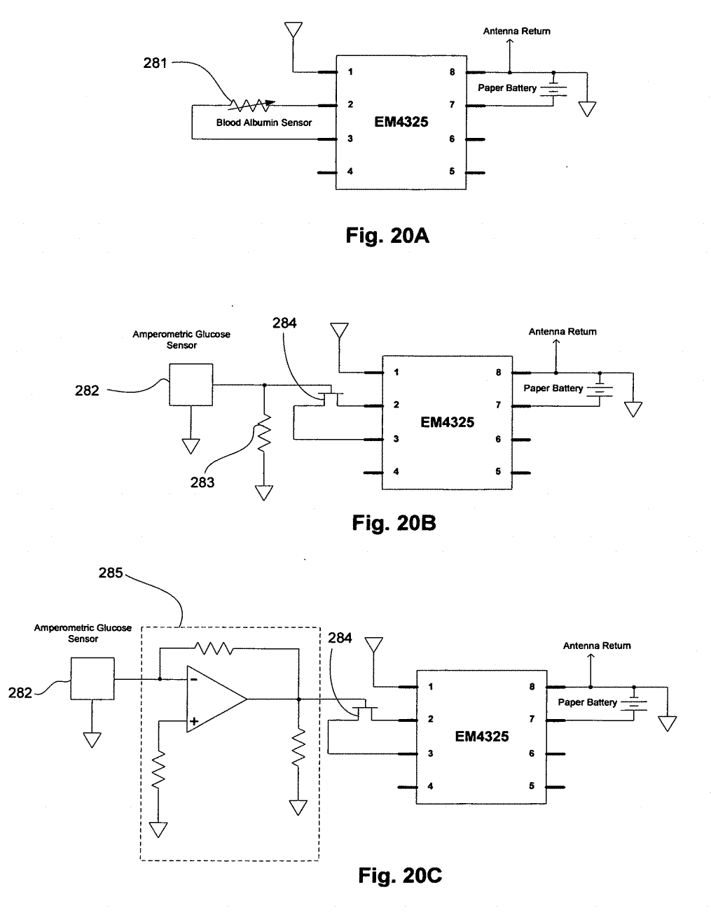

A preferred transponder embodiment uses a highly integrated RFID sensor tag, the EM Microelectronics EM4325. This tag device has a sensor input, is semi-passive, and offers a battery assisted mode of operation with a sensitivity of −31 dBm. It operates in the 860 to 960 MHz ISM band and is inexpensive enough in large quantities to be a disposable component. The battery for this application would be a disposable paper battery from the Israeli company Power Paper Ltd. This technology demonstrates an energy density of 4.5 mA-Hr per square centimeter. FIG. 20A depicts the use of this chip with an impedimetric sensor 281. FIG. 20B depicts use of an amperometric sensor 282, such as a glucose sensor, providing direct current input to a load resistor 283. The resulting voltage signal causes FET switch 284 to enable an alarm condition for the chip. In FIG. 20C, a transimpedance amplifier 285 creates the voltage signal to switch FET 284. Additionally, this tag can be used with energy harvesting chips. International patent application WO 2014/013439 Al by Loussert discloses the use of one EM4325 as an energy harvester for a second EM4325 chip. Other dedicated energy harvesting chips can be used. Further, a dual frequency approach can be used with one frequency to power tag (preferably at a frequency with reduced path loss, but yet with adequate energy intercept by the antenna), the other for communication. Conventional RFID interrogator or readers that meet the full EPC GEN 2 specification can be used to program and interrogate the EM4325. An example of a compact reader is the ARETE POP RFID dongle reader that can plug into a smart phone such as an Apple Iphone or Google Android device. However, its price may be prohibitive for this system, so a low cost reader (parts cost of several few dollars) can be designed and built as discussed below using a few highly integrated semiconductor parts with astute transmitter-receiver isolation design.

### Example Implementations and Link Budget

Reference is made to channel propagation models for body surface to body surface non-line-of-sight (NLOS) propagation. These models have been developed by the IEEE Working Group for Wireless Personal Area Networks (WPANs) to address the needs of Body Area Networks development.

The document “IEEE P802.15-08-0780-09-0006” summarizes the activities and recommendations of the channel modeling subgroup of IEEE802.15.6 (Body Area Network). This guidance is developed for Body Area Networks relating to medical and non-medical devices that could be placed inside or on the surface of human body. The results of theoretical studies and measurement campaigns are provided therein. Path loss and fading models resulting from this work are summarized in the table below for candidate transmission frequency bands comprising those that are unlicensed. Models are provided for the second harmonic frequencies as well. Channel calculations based on the models are used herein for the determination of link margins associated with various network configurations comprising a body-borne sensor/transponder and separate interrogator.

| Frequency | Path Loss Model (dB) | Small Scale Fading |
|---|---|---|
| 13.56 MHz | 20*log10(d) − 4.9 | |
| | (approximately free | |
| | space propagation) | |
| | d in m | |
| 950-956 MHz | 15.5*log10(d) + 5.38 + n; | Ricean |
| (915 MHz | d in mm | K_{dB} = 40.1 − |
| band) | n: Zero mean Gaussian | 0.61*P_{dB} + 2.4*n_{K} |
| | random variable | n_{K}: Zero mean |
| | with σ_{N} = 5.35 | and unit variance |
| | | Gaussian |
| | | random variable |
| 1830 MHz | 3 dB better | |
| | than at 2.4 GHz | |
| 2.4 GHz | 6.6*log10(d ) + 36.1 + n; | Ricean |
| | d in mm | K_{dB} = 30.6 − |
| | n: Zero mean Gaussian | 0.43*P_{dB} + 3.4*n_{K} |
| | random variable | n_{K}: Zero mean and |
| | with σ_{N} = 6.8 | unit variance |
| | | Gaussian |
| | | random variable |
| 4.8 GHz | 19.2*log10(d ) + 3.38 + n; | |
| | d in mm | |
| | n: Zero mean Gaussian | |
| | random variable | |
| | with σ_{N} = 4.4 | |

At HF frequencies, the on-body propagation behavior is close to that of free space. At UHF frequencies, the path loss follows an exponential decay around the perimeter of the body as lossy surface wave propagation comes into effect. This leads to dramatic increase in path loss compared to free space propagation. The loss flattens out for large distance due to the contribution of multipath components from the indoor environment.

The table below summarizes key link parameters that are used in calculating the link margin for the various communication implementations and frequency bands considered in the examples:
OOK modulation with harmonic transponder
FSK modulation with long PN code using harmonic transponder
Chirp modulation with SAW compression transponder

Receiver sensitivities are for low data rates. High Frequency (13.56 MHz) is excluded because favorable propagation loss is overcome by very low antenna efficiency, on the order of −50 dB for a small loop antenna. Further, NFC mode of employment at this frequency permits an interrogator-transponder separation of no more than a few inches.

| | Frequency | | | |
|---|---|---|---|---|
| Parameter | 915 MHz | 1830 MHz | 2.4 GHz | 4.8 GHz |
| Interrogator TX | 0 | | 1 | |
| Antenna Gain (dB) | | | | |
| Interrogator TX | −1.25 | | −1.25 | |
| Antenna Efficiency (dB) | | | | |
| Interrogator RX | | 0 | | 0 |
| Antenna Gain (dB) | | | | |
| Interrogator RX | | −1.25 | | −1.5 |
| Antenna Efficiency (dB) | | | | |
| Transponder TX | 0 | | 1 | |
| Antenna Gain (dB) | | | | |
| Transponder TX | −1.25 | | −1.25 | |
| Antenna Efficiency (dB) | | | | |
| Transponder RX | | 0 | | 0 |
| Antenna Gain (dB) | | | | |
| Transponder RX | | −1.25 | | −1.5 |
| Antenna Efficiency (dB) | | | | |
| One Way Path Loss | 42 | 45 | 52 | 49 |
| (Including Fading) (dB) | | | | |
| Interrogator TX Power (dBm) | 20 | | 1 | |
| Interrogator RX | −110 | −104 | −110 | −110 |
| Sensitivity (dBm) | | | | |
| Process Gain (dB) | | | | |
| FSK PN Code | | 50 | | 50 |
| SAW Pulse Compression | | 40 | | 40 |
| Harmonic Conversion | | 10 | | 10 |
| Loss (dB) | | | | |

Link margins for various system implementations are provided in the table below assuming an on-body propagation distance of several inches.

| | Frequency | | | |
|---|---|---|---|---|
| | 915/1830 | 915 | 2.4/4.8 | 2.4 |
| System Configuration | MHz | MHz | GHz | GHz |
| OOK with 2^{nd} | 10 dB | | 7 dB | |
| harmonic transponder | | | | |
| PN coded FSK with 2^{nd} | 60 dB | | 50 dB | |
| harmonic transponder | | | | |
| Chirp modulation | | 23 dB | | 15 dB |
| with SAW compression | | | | |
| RFID sensor tag | | | | |
| Active | | 30 dB | | 20 dB |
| Passive | | 15 dB | | 10 dB |

### Beacon and Beacon Receiver Implementation

In contrast to the transponder and interrogator implementation, the use of a beacon will involve only one-way communication, from beacon to a beacon receiver. The beacon may initiate a transmission only upon sensor detection of blood or can beacon periodically with a blood detection status. Various commercially-available transmitter chips that operate in the unlicensed ISM bands and exhibiting significant levels of integration, can be used to implement the beacon functionality and are likely more cost-effective than use of discrete components. Among examples are MICRF113 by Micrel, ADF7012 by Analog Devices, or Si4012 (or Si4010) by Silicon Labs. Typically, these types of devices use a digital sensor interface such as a general purpose I/O input. Associated receiver chips include MICRF010, ADF7020, and Si4355. Chip transmitters can provide transmit powers up to +20 dBm, and chip receive sensitivities go down to −126 dBm, thereby providing a maximum link budget of 146 dBm. Given the impetus to use disposable thin batteries, there is sufficient margin to reduce the transmit power to perhaps between −10 to −30 dBm. Even with data that suggests path loss, antenna mismatch, and fading budgets could approach a combined value of 70 dB, use of a −30 dBm transmitter could result in a 20 dB link margin.

The rolling code encoders used in garage door openers can be used to encrypt transmissions from the beacon. A premier device for this purpose is a Microchip KEELOQ R Code Hopping Encoder, such as the HCS300 part.

### Preferred Embodiment Implementation

### Transceiver Approach—Bluetooth LE

Bluetooth Low Energy offers a number of features that are advantageous for the presently disclosed system, i.e. use of a widely adopted standards (IEEE and Bluetooth Special Interest Group), low power consumption, security (including pairing and bonding of devices), low cost chip implementations, and widespread embedding in consumer devices including smart phones.

Performance Indicates very low battery drain for a sensor reporting application analysis (C. Gomez, J. Oiler, and J. Paradells, “Overview and Evaluation of Bluetooth Low Energy: an Emerging Low-Power Wireless Technology,” Sensors, 2012, 12, 11734-11753). In the cited analysis, a sensor-connected transceiver (slave) responds to polling from the master transceiver at fixed intervals. The following link parameters are assumed:

Transmit power set to 0 dBm (5 mA current drain)

Antenna gain of special body-mounted antenna =0 dB (with associated propagation attenuation corresponding to free space behavior)

Receiver sensitivity=−70 dBm

Substantial link margin is available to connect the body worn transceiver with a Bluetooth-enabled smart phone carried in a purse some meters away owing to the special antenna (discussed below regarding antenna technology) that overcomes body-related attenuation.

Energy consumption was calculated during the following states: device wake up, radio turn on (in order to receive the initial BLE packet from the master), request reception, radio switch to transmit mode, notification transmission, and final post-processing before the device returns to sleep mode. For a 32 second interval between sensor readings (completely adequate for the blood detection reporting application), a 4 mAhr capacity paper battery would provide 12 weeks of operation! Ancillary functions such as sensor signal conditioning and interface with the BLE transceiver will consume additional power, but can be kept sufficiently low for the presently disclosed system.

### Transponder Approach

The transponder is the EM4325 device operated in battery assisted passive (BAP) mode thereby achieving a sensitivity of −31 dBm in the 900 MHz ISM band. The power source is a printed paper battery exhibiting a capacity of 4.5 mAhr per square centimeter. The blood sensor is used to create a change from low impedance to high impedance between device pins 2 and 3, thereby signaling an alarm condition. The interrogator is a simplified RFID reader for this device comprising a UHF front end and microcontroller that implements a simplified read protocol. Specifically, a transmitter chip would be combined with a separate receiver chip in order to implement the full duplex TOTAL (Tag Only Talks After Listening) protocol of the ISO 18000 (RFID Air Interface Standards) specification. A number of commercially-available, inexpensive parts exist for the 900 MHz ISM band.

In this reduction to practice, the interrogator periodically sends a CW burst to the transponder which is in low power listening mode. The transponder modulates the reflected power which is returned back to the interrogator. The interrogator demodulates information concerning part identification, and blood sensor status. Because the TOTAL protocol involves full duplex communication with the passive tag, considerable effort must be expended to overcome the transmitter power leakage into the receiver of the interrogator.

A maximum transmitter power of +30 dBm with a receiver sensitivity in the vicinity of −70 dBm is achievable. This is feasible using a circulator or directional coupler to limit transmitter leakage into the and receiver connections to a single antenna and using transmitter and receiver chips with their respective support discrete components in fully shielded, grounded metal enclosures. Also, a bistatic antenna configuration would provide a measure of transmitter-receiver isolation. Additionally, a transceiver implementing the full EPCglobal Class 1 Generation 2 (ISO 18000-6C) specification can be fabricated using a receiver chip, transmitter chip, isolator, and microcontroller, with or without a frame decoder chip.

Various forms of blood sensors can be interfaced with the EM4325 to render an alarm condition upon blood detection, including those types previously discussed. The peptide bonded MWCNT coated fabric sensor presents a nominal low impedance and upon blood contact, a high impedance. Depending on impedance swings indicative of blood detection, it may be required to convert the impedance signal to a voltage signal for input to the GPIO ports of the EM4325. Amperometric sensors would require transimpedance output of a voltage signal also.

### Second Example

An example beacon system comprises use of a low cost, low current transmitter such as the Si4012. The transmitter chip would require minimal circuitry for interface to impedimetric, amperometric, or potentiometric sensors. A good candidate beacon receiver is the Si4362 with a sensitivity of −126 dBm.

Sensor Interface with Communication Devices

For amperometric sensors, typically a transimpedance amplifier is used to convert the current signal to a voltage signal. For impedimetric sensors, a factor of ten increase in impedance swing is achievable with an impedance multiplier circuit as is well known in the prior art. The impedance variation can be transduced to a voltage signal using a Wheatstone bridge or voltage divider with instrumentation amplifier.

Alert-type sensor outputs which are indicative of blood presence will be signals that broach a threshold. If they are current or impedance signals, they can be transduced to voltage signals for direct analog or digital input to transmitter ICs or may be used as switching signals to enable power to the transmitter IC through a FET switch.

Quantitative-type sensor outputs as might be characteristic of LOC blood sensors, might require analog-to-digital conversion for telemetry to a remote receiver.

### Battery Technology

The body mounted sensor can use passive, semi-passive, or active RFID tag technology. Alternatively, low power beacon may be used. In any event, the low duty cycle and limited-use communication from the body will require limited energy that can be supplied by disposable battery or ultracapacitor technology.

Various environmentally-safe, disposable, paper and cloth battery technologies are commercially available to power the disclosed body-mounted sensor. Examples include cloth batteries from FlexEL, LLC of College Park, Maryland with an energy density of 20 milliamp-hour/cm² and paper batteries from Vendum Batteries, Inc. of El Segundo, Calif., Power Paper, Ltd. of Israel, offering typical energy densities of 4 to 5 milliamp-hour/cm².

Scientists at Nanyang Technological University (NTU) in Singapore, Tsinghua University in China, and Case Western Reserve University (CWRU) in the USA claim to have developed a fiber supercapacitor that can be woven into clothing and power wearable medical monitors and communications.

The device packs an interconnected network of graphene and carbon nanotubes so tightly that it stores energy comparably to some thin-film lithium batteries. The product's developers believe the device's volumetric energy density is the highest reported for carbon-based microscale supercapacitors to date—6.3 microwatt hours per cubic millimeter.

### Antennas

Critical to the functionality of the presently disclosed system are the antennas that will provide efficiency in a small physical footprint. The significant research and development devoted to compact WiFi and RFID antenna designs including electrically small, metamaterial, and chip antennas is considered. Disparate technologies can be employed for the interrogation transceiver and the transponder unit, respectively, given cost considerations. Candidate approaches will provide close to 80% radiation efficiency, good impedance matching to target impedance values, and omni-directional patterns, or switched patterns exhibiting gain for diversity purposes. In the interrogator, switching among two or more antennas exhibiting complementary patterns having gain can increase the link margin at minimal additional system cost. In this way, optimal gain will be achieved along the line-of-sight to the transponder in multiplexed fashion. Chip antennas might be more appropriate for the reusable interrogator given cost considerations, whereas printed, disposable antennas would be appropriate for the transponder. An exemplary chip antenna technology is demonstrated by the company Fractus of Barcelona, Spain. The fractal-based designs in this company's product line exhibit omnidirectional, low gain patterns, low VSWR, and greater than 70% efficiency in bands of relevance. For the transponder, a thin wire antenna can be bonded to the perimeter of the blood-absorbing pad or an antenna can be printed on paper or fabric for inclusion in the pad in a geometry that maximizes the length of the antenna.

Many antenna designs can be rendered by inkjet printing of conductive inks on paper or flexible film polymeric substrates. A good example of a multi-band design that can be rendered as a printed antenna is provided by C.-T. Lee, S.-W. Su, and F.-S. Chang, “A Compact, Planar Plate-Type Antenna for 2.4/5.2/5.8-GHz Tri-Band WLAN Operation,” Progress In Electromagnetics Research Letters, Vol. 26, 125-134, 2011. The antenna is 10 millimeters wide and 37 millimeters in length. Some tuning of this design could provide operation at 5 GHz.

At UHF frequencies, when the tag is placed on high dielectric (human) or high conductivity (metals) surfaces, the performance of the tag is degraded. These surfaces affect the electromagnetic behavior of the tag and hence the read range performance of the tag. Antennas mounted on metal or water (body) face challenges that must be accommodated to prevent pattern, impedance, and efficiency variations. Various designs have been developed for body-mounted antennas. A premier UHF design is found in the paper to Rajagopalan et al. (H. Rajagopalan and Y. Rahmat-Samii, “Conformal RFID antenna design suitable for human monitoring and metallic platforms,” in Proceedings of the 4th European Conference on Antennas and Propagation (EuCAP '10), pp. 1-5, Barcelona, Spain, April 2010.) Disclosed is a conformal RFID tag for remote human monitoring and metallic cylinder tracking. This antenna exhibits only a few dB variation in gain between a free space connection and body-mounted connection.

Retrodirective antennas with only a few array elements can achieve significant effective antenna gain as in U.S. patent application number 20120001735 to Fink, et al. Also, a structure separate from the main body-worn antenna, could take the form of a reflector antenna that is also mounted on the body to increase received signal strength in the vicinity of the main antenna. Noteworthy, is the plasmonic tag technology of Omni-ID of Forster City, Calif. which uses plasmons formed in the receiving surface to concentrate energy for return.

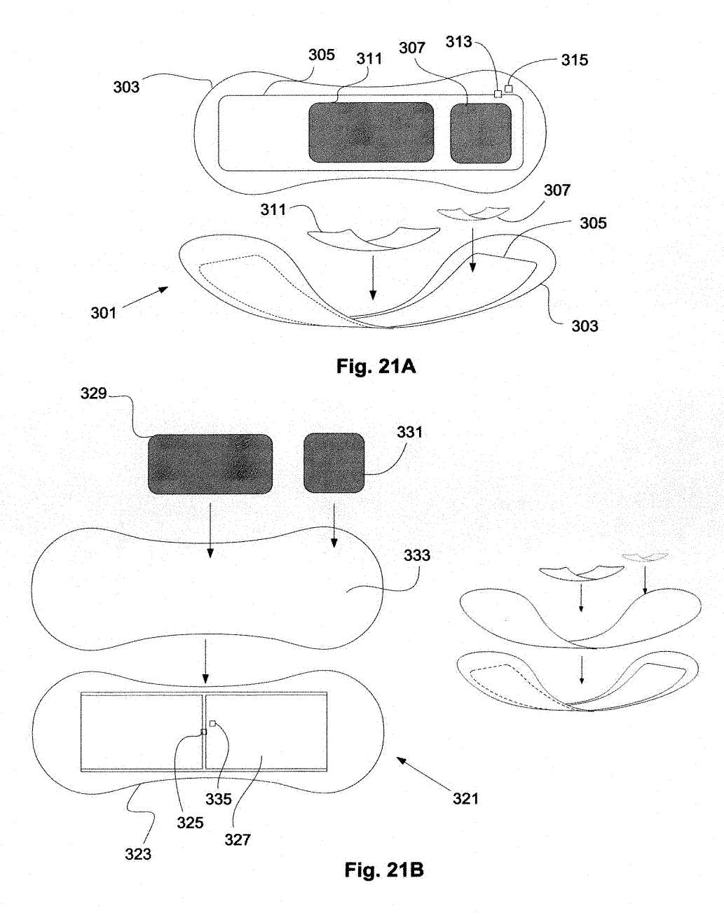

Reference is made to FIGS. 21A and 21B, depicting pantyliner-based sensors 301 and 321, respectively, exhibiting different antenna configurations. In FIG. 21A, a simple loop antenna 305 is shown connected to communication chip 313 (either a transceiver chip such as a Bluetooth chip, a passive/semi-passive tag chip, or an active beacon chip). Sensor interface electronics 315 is electrically connected (not shown) to blood detection sensor 311 and provides control or annunciation input to chip 313. A disposable, flexible battery 307 supplies power and any needed bias voltages. All components are mounted on the pantyliner 303. Antenna 305 and electrical connections can be printed on the fabric of the pantyliner 323. Use of a specially-designed body mount antenna (after the reference above to Rajagopalan et al.) 327 is shown in FIG. 21B. The communication chip 325 is mounted at the antenna feed point adjacent to sensor interface electronics 335. An insulating layer 333 forms the substrate for the blood sensor 329 and mounting of the disposable battery 331. Qualcomm has developed a disposable body-mountable antenna applique that maintains antenna gain in proximity to the body and overcomes the body loss attenuation associated with other antenna types; this applique includes a paper battery (from Enfucell) and a Bluetooth LE transceiver-based chip. Such a combination can comprise one layer of the pantyliner sensor 321 or can be a separate applique that is electrically connected to the pantyliner 321 blood detector. Configuration options for use of this technology include a) a wholly disposable pantyliner sensor comprising within the thin pantyliner, a blood detector, electronics for interface to a Bluetooth transceiver, a Bluetooth transceiver, and the antenna, b) a disposable pantyliner containing blood detection sensor with a reusable (if only reusable for the episode required for menstrual onset sensing) separate Bluetooth transceiver, antenna and battery that is conveniently attachable to the pantyliner, c) disposable pantyliner sensor comprising blood detector and disposable battery along with reusable Bluetooth transceiver and sensor interface electronics.

Electrical connections on these disposable substrates whether comprising fabric or paper, can be achieved with printed conductors. The mating of disposable and reusable portions of the system can be done with simple adhesive connections. Combinations of the transceiver and battery portions of the system can be reused for a period of time before disposal.

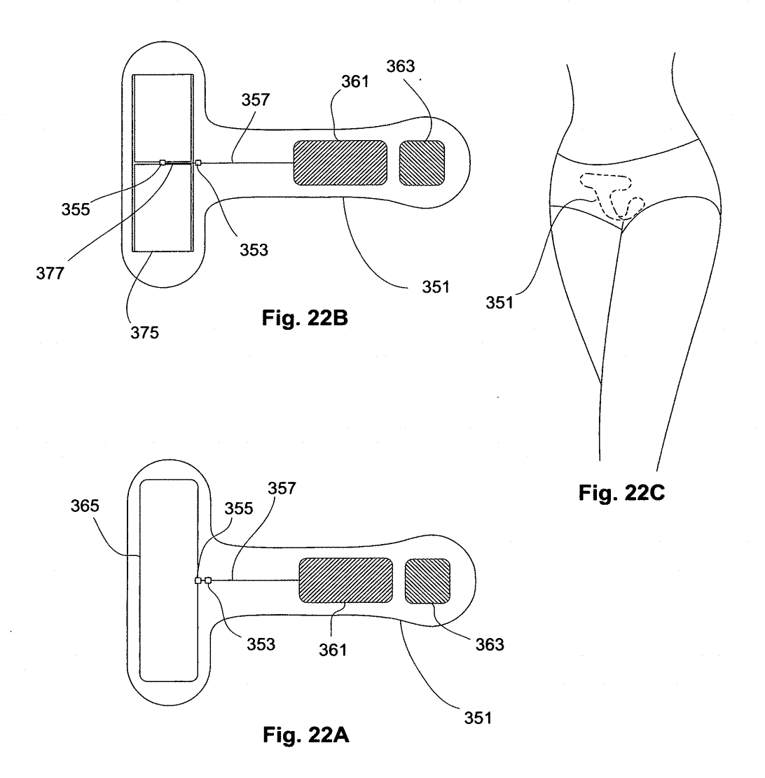

Provision for an extended antenna geometry is featured in FIG. 22. In both FIGS. 22A and 22B, the pantyliner 351 is shown to be elongated and with a “T” section at one end to accommodate horizontal placement of the antenna. In FIG. 22A, the loop antenna 365 is connected to communication chip 355 which in turn has connection to the blood sensor 361 through interface electronics 353. Disposable battery 363 is placed adjacent to the sensor. In FIG. 22B, the antenna 375 of Rajagopalan et al. is shown with the communication chip 355 mounted at the antenna feedpoint. The horizontal placement of the antenna with the pantyliner 351 in the appropriate undergarment position is shown in FIG. 22C.

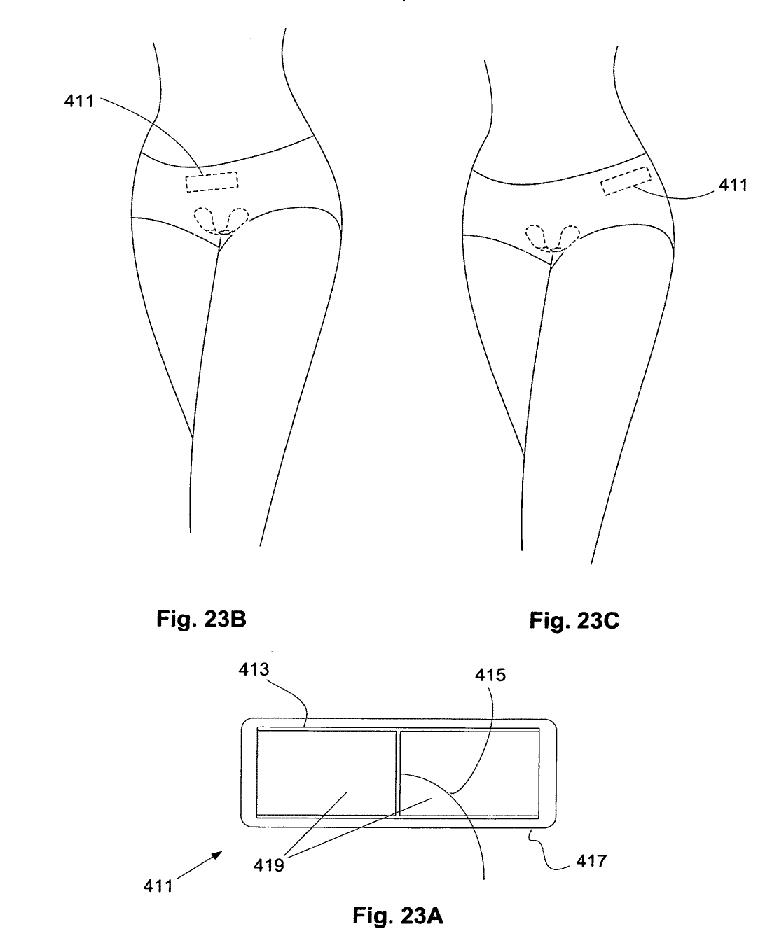

Alternatively, the antenna can be rendered in an applique 411 separate from the pantyliner as shown in FIG. 23A. Any number of antenna designs can be implemented for this purpose. As an example, the antenna 413 of Rajagopalan et al. is shown mounted to an adhesive substrate 417 for installation in an undergarment. This antenna is characterized by two end-shorted patches 419 with a feed point between them shown connected to a notional conductor path 415 that would connect to the pantyliner electronics. Different options for antenna mounting locations are shown in FIGS. 23B and 23C.

### Annunciation of Blood Detection

The annunciation should be covert given the constraint that the user may be in public at the time of annunciation. For covert annunciation, two chief categories of alert exist, skin contact sensation and RF communication with an audible or visual indicator. The annunciation may be audible and still remain covert if, for instance, the alert is associated with a ring tone on a smart phone. With respect to skin contact, temperature and vibration are leading prospects for sensory stimulation. Since temperature sensitivity of the skin is modulated by ambient temperature, the favored approach will be to use a vibrating actuator; the peak vibration sensitivity of the body occurs around 250 Hz. A small electromechanical transducer such as a piezoelectric disk can be used to provide such vibratory stimulus, avoiding an acoustic signature that might be apparent to others than the user.

Alternatively, the detection of blood can be annunciated to the user by annunciation associated with a dedicated interrogator generating a ring tone, as described above, or by means of a low power RF connection to a smart phone, tablet device, or other communications or PDA appliance. For example, a low power Bluetooth connection with a smart phone can provide annunciation appropriately coded. For alerts during sleep, the annunciation can be made appropriately intense or provocative. The Bluetooth Special Interest Group is working to extend the “Health Device Profile” software protocols to Bluetooth Low Power. This will facilitate Bluetooth Low Power use with a host of personal medical sensing devices.

When communicated via RF link (or entered manually) to a smartphone or tablet application, this data concerning menstrual onset may be used in conjunction with basal temperature to more accurately predict ovulation. Many iPhone and Android applications already exist that accept these data. The benefit here would be more accurate timing of menstrual onset and duration, and possibly automatic data entry via Bluetooth. This device can communicate with a wrist worn appliance such as the Apple Watch via an RF link.

### Smart Bandage

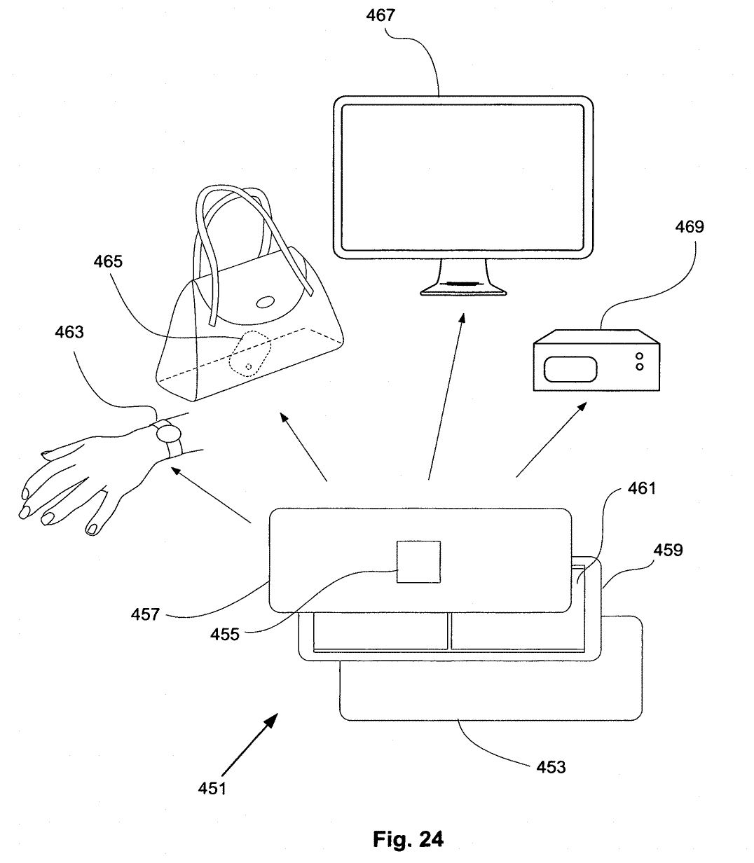

A body-worn device 451 comprising blood sensor, associated communication electronics, and battery, communicating information to a receiver or transceiver on the body, or remote to the body, is depicted in FIG. 24. The device 451 can represent the previously described pantyliner insert for menstruation or a smart bandage. In the case of a smart bandage, device 451 telemeters wound and skin health-related information to a separate receiver or transceiver. The device 451 is shown with three functional layers, a sensor substrate 457 containing the sensor 455, an antenna layer 459 with contained antenna 461, and an adhesive layer 453 that serves to seal the composite device to the skin. Also, memory and processing functions can be included in the electronics resident in the device as a bandage.

Radiofrequency electronics or infrared transmitter (not shown) are contained in the device for communicating health information to various candidate receivers. These include a body worn receiver (or transceiver), as depicted in a wrist-worn embodiment 463, a receiver (or transceiver) module 465 shown carried in a purse, a remote data logging receiver (or transceiver) 469, and a nurses' station display 467.

The sensor can be an LOC implementation or other sensor type that can sense various wound and skin health-related parameters. For example, detection of pathogens or disease conditions, deleterious wound states, perspiration markers, blood, etc. (D. Liana, et al., “Recent Advances in Paper-Based Sensors,” Sensors 2012, 12, 11505-11526; doi: 10.3390/s120911505, Wang, et al., “Integration of cell phone imaging with microchip ELISA to detect ovarian cancer HE4 biomarker in urine at the point-of-care,” Lab on a Chip, Issue 20, 2011.).

1. A system for detection of menstrual spotting that precedes menstrual flow, said system comprising:
a) a real-time, relatively thin, wide area blood detection sensor, and
b) a first radiofrequency communicator electrically connected to the real time area wide blood detection sensor,
the real time area wide blood detection sensor providing an indication of first menstrual spotting over an area in the vicinity of normal menstrual emanation, the first radiofrequency communicator capable of communicating a blood detection signal from the real-time, wide area, blood detection sensor to a second radiofrequency communicator which further is connected to an annunciator for annunciation of the blood detection to a user of the system.

2. A system for detection of menstrual spotting that precedes menstrual flow, as recited in claim 1 wherein the first radiofrequency communicator and the second radiofrequency communicator are both transceivers.

3. A system for detection of menstrual spotting that precedes menstrual flow, as recited in claim 2 wherein the real time, wide area, relatively thin blood detection sensor and the first radio frequency transceiver are contained in a pantyliner insert.

4. A system for detection of menstrual spotting that precedes menstrual flow as recited in claim 2 wherein the real-time, wide are blood detection sensor discriminates blood from among blood, other vaginal secretions, urine, and perspiration.

5. A system for detection of menstrual spotting that precedes menstrual flow as recited in claim 4 wherein the real-time blood detection sensor detects a marker taken from the group comprising blood albumin, hemoglobin, fibrinogen, and any one of 385 menses-specific blood markers.

6. A system for detection of menstrual spotting that precedes menstrual flow as recited in claim 2 wherein the real-time, wide are blood detection sensor additionally discriminates blood from among blood, water, and spilled beverages.

7. A system for detection of menstrual spotting that precedes menstrual flow as recited in claim 2 wherein the blood detection sensor comprises an electrically conductive fabric containing anti-human albumin antibody treated carbon nanotubes.

8. A system for detection of menstrual spotting that precedes menstrual flow as recited in claim 2 wherein the blood detection sensor comprises a fabric containing electrically conductive graphene exhibiting change in electrical impedance in the presence of blood proteins.

9. A system as recited in claim 2 wherein the blood detection sensor spans a contiguous area of few square inches and is attachable to underwear or a light weight menstrual pad.

10. A system as recited in claim 2 wherein the blood detection sensor resides at the periphery of a conventional menstrual pad for the purpose of detecting blood broaching the edge of the menstrual pad.

11. A system as recited in claim 2 which includes memory to store the times of menstrual onset, and a processor that makes use of a software application to process the times of menstrual onset to predict ovulation.

12. A system as recited in claim 2 wherein the blood detection sensor modifies the response of a surface acoustic wave device.

13. A system for detection of menstrual spotting that precedes menstrual flow, as recited in claim 1 which further comprises:
a) the second radiofrequency communicator, and
b) the annunciator.

14. A system for detection of menstrual spotting that precedes menstrual flow, as recited in claim 13 wherein the first radiofrequency communicator is a transponder and the second radiofrequency communicator is an interrogator.

15. A system for detection of menstrual spotting that precedes menstrual flow, as recited in claim 14 wherein the radio frequency interrogator interrogates the radio frequency transponder with a signal at given frequency and the transponder returns a signal at a harmonic of the given frequency, the return signal amplitude of sufficient indicative of the presence of blood on the sensor.

16. A system as recited in claim 14 wherein the annunciator comprises a vibrating actuator for covert alert of the user.

17. A system as recited in claim 14 wherein the annunciator comprises a radio frequency relay of the blood detection to a remote device.

18. A system as recited in claim 14 wherein the radio frequency interrogator relays a blood detection signal to a smart phone or PDA device.

19. A system for detection of menstrual spotting that precedes menstrual flow, as recited in claim 13 wherein the first radiofrequency communicator is a beacon and the second radiofrequency communicator is a beacon receiver.

20. A method of detecting menstrual spotting that precedes menstrual flow and annunciating the detection to the individual anticipating menstrual flow, the method comprising:
a) providing a wide area, thin blood sensor,
b) providing a transceiver in electrical connection with the blood sensor,
c) providing a body-mountable antenna in electrical connection to the transceiver,
d) providing a disposable battery in electrical connection with the transceiver and blood sensor,
e) configuring the blood sensor as part of a pantyliner insert,
f) placing the pantyliner insert in an undergarment,
g) sending a signal from the first transceiver to a remote second transceiver if blood is detected by the blood sensor, and
h) annunciating the detection of blood.
